(19) United States
(12) Patent Application Publication
(10) Pub. No.: US 2003/0163846 A1
(43) Pub. Date: Aug. 28, 2003

(54) Process for the genetic modification of a plant

(76) Inventors: John M. Ward, Falcon Heights, MN (US); Andreas Weise, Freiburg (DE); Laurence Barker, Tubingen (DE); Wolf-Bernd Frommer, Tubingen (DE); Waltraud Schulze, Odense (DK); Christina Kuhn, Tubingen (DE)

Correspondence Address: S. Peter Ludwig. Esq., DARBY & DARBY P.C., 805 Third Avenue, New York, NY, 10022-7513, US

(21) Appl. No.: 10/247,813

(22) Filed: Sep. 18, 2002

Related U.S. Application Data
(63) Continuation of application No. PCT/EP01/02148, filed on Feb. 26, 2001.

(30) Foreign Application Priority Data
Mar. 24, 2000 (DE) DE 100 14 672.4
Oct. 11, 2000 (DE) DE 100 50 233.4

(51) Int. Cl. A01H 5/00; C12N 15/82; C07K 14/415

(52) U.S. Cl. 800/284; 435/468; 435/419; 530/370

(57) ABSTRACT

The invention relates to nucleic acid molecules that code a saccharide transporter, in particular a saccharose transporter, vectors and host cells that contain said nucleic acid molecules, as well as plant cells and plants transformed by the described nucleic acid molecules and vectors. The invention also relates to processes for modifying the transport of saccharide, in particular saccharose, in plants.

### DESCRIPTION

This invention relates to nucleic acid molecules that code a saccharide, in particular saccharose transporters, vectors, and host cells that contain these nucleic acid molecules, as well as the fungi, plant cells, and plants transformed using the nucleic acid molecules and vectors described herein. The invention further relates to processes for modifying saccharide transport and in particular saccharose transport in plants.

Higher plants have heterotrophic tissue that is supplied with carbohydrates by autotrophic tissue. Saccharose and its derivatives are the main form in which carbohydrates are transported. The heterotrophic tissue is supplied via the phloem, which connects to the organs that supply excess amounts of photoassimilates, in other words carbohydrates, and that can export these photoassimilates—in other words so-called source organs—with organs that, in their net balance, must import photoassimilates, in other words so-called sink organs. Source organs are, for example, mature leaves and sprouting seeds. Sink organs are, for example, young leaves, young tubers, roots, fruits, blossoms, and other reproductive organs. The phloem is constructed of various cell types such as sieve elements, cap cells, parenchyma cells, and bundle sheet cells. In the sieve elements the translocation flux of the photoassimilates moves from export sites to import sites. Both the loading of the phloem with photoassimilates and its unloading can, theoretically, be performed via apoplastic and symplastic routes, and a multitude of factors such as osmotic ratios, concentration gradients, plasma membranes that must be crossed, etc. can affect the loading and unloading. Thus, the loading of the phloem as well as the supplying of the sink organs with photoassimilates are highly complex processes that obviously comprise a multitude of closely interrelated and regulated transport steps. These transport steps take place with the participation of various plasma membrane proteins, in particular transport proteins. For example, various members of the saccharose transporter family—such as SUT1 (sucrose transporter) are known to be found in various families of plants. The SUT genes code hydrophobic proteins that have 12 transmembrane domains and are clearly distinguishable from the members of the hexose transporter family. Lalonde et al. (The Plant Cell (1999) 11, 707-726) leads one to suspect that the members of the SUT family have a high affinity for saccharose. It is assumed, that, in particular, the saccharose transporter SUT1 from Lycopersicon esculentum, Nicotiana tabacum, and Solanum tuberosum is responsible for the long-distance transport in the phloem (Riesmeier et al., Plant Cell (1993) 5, 1591-1598). WO94/00574 discloses the DNA and amino acid sequences of SUT1 from spinach and potatoes. This saccharose transporter, which is located in the plasma membrane of the sieve elements of the phloem, is an essential component for the long-distance transport of saccharose in the phloem, as has been shown, for example, in antisense inhibition experiments in transgenic potato and tobacco plants (Riesmeier at al., EMBO (1994) 13, 1-7) (Lalonde et al., op. cit.). The expression pattern of SUT1, in particular the expression in the entire phloem, proves that SUT1 is responsible for maintaining the concentration gradients of saccharose between the loading and unloading zones (leaves, in other words sink organs). SUT1 appears therefore to be less responsible for the (first) loading of the phloem in source organs and more for the return of saccharose coming from the phloem. This function is further confirmed by the relatively high affinity and the low transport capacity associated with SUT1 . Thus, SUT1 can import very low amounts of saccharose from the apoplast (back) into the phloem, and therefore it keeps the apoplastic concentrations low. Since it functions at these low concentrations, it cannot be responsible for high transport rates. The kinetics of the saccharose intake in leaf blades clearly reveals this high-affinity (Km 2.7 mM) intake combined with low capacity (Vmax 0.7 nmol cm⁻² min⁻¹) (Delrot and Bonnemain, Plant Physiol. (1981) 67, 560-564). This system is also referred to as a HAS (high affinity/low capacity system). The involvement of SUT1 in the entire transport and regulation system of saccharose transport however is completely unclear (Ward et al., Int. Rev. Cytology (1998) 178, 41-71, Kühn et al., J. Exp. Bot. (1999) 50, 935-953). This also applies to the identification of a (first) loading carrier, which is responsible for a second HCS (high capacity/low affinity system) kinetic component that may be present. In addition, it is highly probable that there are large differences in the saccharose transport mechanism between various plant species (Lalonde et al., op. cit.) and the large number of potential regulating mechanism and factors that affect the transcriptional and post-transcriptional level (Kühn et al., Science (1997) 275, 1298-1300). Lalonde et al. (op. cit.) speculate that in addition to the saccharose transporters, additional functional elements are involved in the saccharose transport, for example regulator and sensor elements. The extreme complexity of the saccharose transport mechanism, however, thus far has not allowed structures to be systematically assigned to functions nor their interactions to be predicted in a manner that will permit systematic intervention in the saccharide transport, in particular in the saccharose transport, with the goal of obtaining improved plants in regard to specific characteristics. This is most clearly seen in the fact that an overexpression of SUT1 did not produce any changes in the allocation of assimilates, but only exhibited an influence on blooming characteristics (U.S. Pat. No. 6,025,544; P 44 39 748.8).

Thus, the basic technical problem underlying the invention is to provide processes and means for preparing a plant modified by means of genetic engineering that permit controlled intervention in the saccharide transport fluxes, in particular the saccharose transport fluxes, of the plant in such a way that a plant having improved characteristics, for example increased sugar contents in sink organs, in particular in harvest organs, can be created.

The invention solves this problem by providing a process for modifying the saccharide flux and/or the saccharide concentration, in particular the saccharose flux and/or the saccharose concentration, in the tissues of a plant such that a modified activity of the saccharide transporter having a low saccharide affinity but a high saccharide transport capacity results in the plant. The teachings of the inventions also provide for the first time means and processes for systematically influencing the saccharide flux and the saccharide concentration in the various tissue of the plant by modifying the activity of a saccharide transporter having a low saccharide affinity and a high saccharide transport capacity—in other words to modify it such that, in the course of this process, a modified plant is produced. The present invention for the first time provides an HCS system—in other words a saccharose transport system that has a high transport capacity for saccharose but low affinity to saccharose—that, in particular, is expressed in the micro veins of a plant.

With regard to the present invention, a modification of the activity of the saccharide transport, in particular of a saccharide transporter, is understood to mean a change in the normal activity relative to the wild-type activity—for example, a complete suppression, reduction, or increase in activity. This increase in activity can be attributed to a modified activity of the protein itself, caused, for example by posttranscriptional modifications such as phosphorylations or dephosphorylations. However, it can also be caused by a modified expression rate of the coding gene, a modified stability or translation rate of the mRNA that is formed, or for some similar reason, and thus also by a change in the amount of the saccharide transporter present in a tissue. The modification of the activity of the saccharide transporter may also be due to the modification of the activity of an element that controls or regulates the activity of the saccharide transporter—for example, a sensor or regulator protein.

In a preferred embodiment, the saccharide is understood to mean saccharose and the saccharide transporter is understood to mean a saccharose transporter, unless otherwise stated SUT4 (sucrose transporter 4)—in other words, a transporter having a low affinity to saccharose and a high transport capacity for saccharose.

Slight or low affinity as understood in the context of this invention is understood to mean an affinity that is less than the SUT1 affinity—for example about 50% preferably 80%, 100%, 200%, 300%, or 400% below that of SUT 1—for example, an affinity Kₘ>2.7 mM, preferably >4 mM, >6 mM, >8 mM, >10 mM, >15 mM, >20 mM and preferably >25, more preferably 26 mM. A high transport capacity is understood in the present invention to mean a transport capacity that lies above the transport capacity of SUT1, for example 50%, 100%, 150%, 200%, 300%, 400%, or 500% above that of SUT1. A transport capacity Vₘₐₓof >0.7, preferably >1,>1.5,>2,>3,>3.5, more preferably 3.6 nmol cm⁻² min⁻¹.

The invention is based on the discovery and the teachings derived therefrom that technical means, in particular genetic engineering means, can be used to influence an activity of the said saccharide transporter, which may only be present endogenously, or to introduce such an activity into a plant.

With regard to the present invention, the term sink organs is understood to mean organs or tissue of plants that, in terms of their net balance, must import photoassimilates. Such plant organs or tissues are young leaves, tubers, fruits, roots, blossoms, reproductive organs, wood, support tissue, buds, seeds, bulbous roots, etc.

With regard to the present invention, the term source organ is understood to mean organs or tissues of plants that, relative to their net balance, have excess amounts of photoassimilates and can export said photoassimilates. Source organs are, for example, mature leaves and sprouting seeds, tubers, and bulbs.

In the invention it was demonstrated that an increased expression rate of the saccharide transporter modified in accordance with the invention, and an increased transport rate, respectively, for example in cap cells and/or sieve elements substantially increases the saccharide loading of the phloem, in particular the saccharose loading of the phloem, in particular at a high light intensity or high CO₂ concentration, by which means plants having harvest organs that exhibit an increased saccharose content can preferably be obtained. The invention solves the problem in particular by providing a process for modifying the saccharide flux and/or the saccharide concentration in the tissues of a plant, in which the activity of a saccharide transporter having a low affinity to saccharide and a high transport capacity for the saccharide is modified by transforming at least one plant cell using at least one vector containing nucleotide sequences that allow the saccharide transporter to be modified, and in which the plant cell containing said nucleotide sequences in a stably integrated form is regenerated to form a plant in whose tissue a modified saccharide flux and/or a modified saccharide concentration is present in each case compared to a wild-type plant, by which is meant a plant that has not been transformed in accordance with the invention. Thus, the invention teaches the modification of a saccharide transporter having a high transport capacity for the saccharide and a low affinity to the saccharide, with such modification being achieved by means of manipulating the plant through the application of genetic technology.

In the invention, this can occur when a plant cell is transformed by at least one vector containing the coding nucleotide sequences of the saccharide transporter, and when the vector permits an overexpression of the saccharide transporter in the transformed tissue after the stable integration of the nucleotide sequences in the genome of the transformed cell.

The overexpression of the nucleotide sequences of the said saccharide transporter that are used in the invention, for example in sieve elements of cap cells, causes an increased saccharose loading in the phloem. Plants produced using the process of the invention have, for example, a higher carbohydrate content in sink organs, for example in roots, fruits, tubers, blossoms, or seeds. An increased carbohydrate content results in a preferred manner, in particular in the harvest organs of the plant, which are frequently sink organs. If the process of the invention is used in an especially preferred embodiment of the invention with oil-containing plants, for example rapeseed, an increased oil content can be observed in the harvest organs. The observation that the number of harvest organs is increased and also that their weight can be increased is especially advantageous.

The overexpression of the saccharide transporter in the aforesaid manner in source organs as provided in the invention may also advantageously cause the blossoming time of the transformed plant to be changed.

Since the transport of sugar in the phloem is frequently coupled to the transport of amino acid in the phloem in a reciprocal manner, the increase of the saccharide content in the phloem as provided for in the invention can also decrease the undesirable amino acid content in sink organs, for example in potato tubers or in the stake root of the sugar beet. Finally, the process of the invention can increase the glycosylation rate of substances that are endogenously present in plants, or also of substances that are applied exogenously to plants such as xenobiotics, for example herbicides or pesticides, and thus increase their mobility.

In an especially preferred embodiment of the present invention, the overexpression in source organs that is described above is achieved by transforming SUT4-coding nucleotide sequences under the control of source-specific promoters, in other words in particular leaf-specific and/or cap-cell-specific promoters, cloned into a vector, in at least one plant cell and, integrating them into the genome of the plant cell, preferably in a stable manner.

In a further embodiment of the invention, constitutively expressing promoters such as the 35SCaMV promoter, the cap-cell-specific rolC promotor from an agrobacterium or the enhanced PMA4 promotor (Morian et al., Plant J (1999) 19, 31-41) can be used.

In an additional preferred embodiment, the modification of the saccharide transport is accomplished by modifying the activity of the saccharide transporter through overexpression in the leaf mesophyll and/or leaf epidermis. The specific expression of SUT4-coding nucleotide sequences in these tissues results in a competitive effect relative to the endogenous saccharose transporter that is active in the sieve elements, so that the carbohydrate content in the leaves is increased. The plants produced in this manner have larger leaves, which incidentally also afford improved protection against pathogens. One of the reasons for this is that genes that have a defense function are activated by an elevated sugar content. In addition, a thicker cuticula and a higher secondary metabolite content result in substances which may be used, among other things, as precursors for the production of biodegradable plastics like PHA (polyhydroxyalcanoates). The expression in the leaf mesophyll and epidermis provided in this preferred embodiment can be achieved through the use in an especially preferred embodiment of the promotor StLS1/L700 (Stockhaus et al., Plant Cell (1989) 1, 805-813), of other epidermis-specific promotors, or of the PFP promotor (palisade-parenchyma) (WO98/18940) to express the SUT4-coding nucleotide sequences.

In an other preferred embodiment of the invention, an overexpression of the saccharide transporter is specifically provided in sink cells or organs, in particular in seeds developing in a plant. This results, among other things, in an improved germination rate, since both the carbohydrate and, in particular the oil content of the seeds are increased.

The tissue-specific promotors whose use is preferred in this embodiment for the expression of the SUT4-coding nucleotide systems in seed tissue are, for example, the vicilin promotor from Pisum sativum (Newbigin et al. Planta (1990) 180, 461-470).

In a further preferred embodiment of the present invention, an aforesaid process is provided in which an overexpression of the saccharide transporter is accomplished by using tissue-specific regulatory elements for the epidermis and parenchyma of sink organs. The increased expression of the saccharide transporter used in accordance with the invention in sink organs increases their ability to take in saccharide and increases the saccharide flux into the sink tissue. Plants produced in accordance with the invention therefore have, for example, larger, more colorful blossoms and/or an increased number of blossoms, larger seeds, larger tubers, or larger stake roots. The sink organs may also have a higher carbohydrate content and a higher oil content, an improved structure, in particular strength, faster growth, and/or optionally improved tolerance to cold based on the higher content of osmotically active substances. In addition, the blooming time and duration as well as the development of fruits can also be influenced.

In an especially preferred embodiment, the invention provides, for the aforesaid overexpression in the sink epidermis and parenchyma the AAP-1 (amino acid permease 1) promotor, for example the arabidopsis promotors AtAAP1 (expression in the endosperm and during early embryonic development), or AtAAP2 (expression in the phloem of the funiculus) (Hirner et al., Plant J. (1998) 14, 535-544), the B33 (patatin) promotor (Rocha-Sosa et al., EMBO J. (1998) 8, 23-29) (access number: X14483; all of the access numbers referred to here relate to the following gene bank, unless otherwise stated: National Center for Biotechnology Information, National Library of Medicine, Bethesda, Md. 20894, USA) in particular for tubers and stake roots, the vicilin (storage protein) promotor from Pisum sativum (Newbigin et al. Planta (1990) 180, 461-470) (access number: M73805) and/or seed and blossom-specific promotors.

The invention also relates to the modification of saccharide transport activity in the tissues of a plant, where the activity of a saccharide transporter, in particular of a saccharose transporter, having a high transport capacity for saccharose and a low affinity to saccharose is suppressed or reduced, in particular inhibited or cosuppressed.

In an especially preferred embodiment the activity of the saccharide transporter can be suppressed or reduced by transforming the plant cells using vectors that have the saccharide transporter-coding nucleotide sequences used in the invention or sufficient portions thereof for an antisense repression in an antisense orientation relative to a promoter, and are preferably integrated in a stable manner into the genome of the plant cell. The expression of this antisense RNA suppresses or reduces the formation of the aforesaid endogenously present saccharide transporter so that the saccharide flux caused by this transporter can be manipulated.

In a further preferred embodiment, the activity of this saccharide transporter can be reduced or suppressed by means of the cosuppression effects introduced into the plant. In order to achieve these cosuppression effects, in the preferred embodiment a plurality of copies of a vector are introduced into at least one plant cell preferably in a stable manner in their genome, said vector containing the saccharide-transporter-coding nucleotide sequence or parts thereof, whereby said copies are integrated in the genome.

In a further preferred embodiment, the activity of the saccharide transporter can be suppressed or reduced by mutagenizing preferably tissue-specific endogenously present nucleotide sequences of the saccharide transporter that is to be used in accordance with the invention, in other words nucleotide sequences that are already present in the non-transformed wild type—for example by means of transposon mutagenesis.

Finally, the invention may be used to reduce or suppress the activity or expression of the saccharose transporter by means of RNA-double-strand inhibition.

In an especially preferred embodiment of the present invention, the aforesaid techniques for suppressing or reducing the activity of the saccharide transporter having a low affinity to saccharose but a high saccharose transport capacity can be used, for example, in source tissues such as leaves to produce a higher carbohydrate content, in particular a higher saccharose content. In a preferred embodiment of the invention, this can be accomplished through the reduction or suppression of the saccharide transport capacity in source organs as described above. This reduces the structure and strength of sink organs while the saccharose or carbohydrate content in source organs increases. By this means, the sweetness of the source organs of certain plants whose leaves are used as food—for example lettuce or spinach—can be increased. In addition, advantages gained through competition can be achieved as described above for the overexpression of the saccharide transporter in the leaf mesophyll and epidermis. Finally, as a result of the reduced saccharose flux in the stem of a plant, its length may be reduced, which is particularly desirable for producing dwarf versions of plants, for example in the case of certain types of apples, etc.

In a further preferred embodiment, the activity of the saccharide transporter in the guard cells can be suppressed or reduced, for example by mutagenesis of endogenously present nucleotide sequences that code the saccharide transporter in guard cells, through cosuppression effects, RNA double-strand inhibition, or through the use of antisense structures. By modifying saccharide transport processes and the changes in the availability of energy and osmotically active substances associated therewith in guard cells, the ability of stomata to open or close can be changed, in particular increased or reduced. A higher stomata opening rate permits the supply of CO₂ to be increased, thus improving the rate of photosynthesis. If the invention is used to inhibit the opening of the stomata or to reduce the frequency of opening, the plant's resistance to drying can be improved. In an especially preferred manner a vector is used to achieve the aforesaid effects. And in this vector the saccharide-transporter-coding nucleotide sequences that are used can be present in a sense or anti-sense orientation, for example controlled by a guard cell-specific promoter, for example the KAT1 promotor (Nakamura et al., Plant Physio. (1995) 109, 371-374).

Since the saccharide transporter SUT4 is also expressed in sink organs, the invention can be used to reduce the importation of saccharide into the sink cells or organs or, preferably, into certain sink cells or organs, more preferably into the blossom. This can be used to cause carbohydrates that are useful for other synthesis paths to accumulate in source cells or organs, and the relative activity of individual sink cells can be shifted in favor of other sink cells, thus qualitatively and quantitatively improving yields.

In an especially preferred embodiment of the invention, the aforesaid processes can be performed and in addition to or instead of the saccharide transporter, preferably saccharose transporter, used in the invention, especially SUT4-coding nucleotide sequences, additional nucleotide sequences can be used for the transformation. These additional nucleotide sequences have a functional relationship with the saccharose concentration and the saccharose flux in the tissues of a plant.

In an especially preferred embodiment, these are nucleotide sequences that code SUT1 or SUT2, for example genomic or cDNA-nucleotide sequences.

SUT1 genomic and cDNA sequences are disclosed, for example, in WO94/00574 (potato, spinach), Riesmeier et al., op. cit. (potato), Riesmeier et al., (EMBO J. (1992) 11, 4705-4713 (spinach)), Bürkle et al., (Plant Physio. (1998) 118, 59-68 (tobacco)), Hirose et al., (Plant Cell Physiol. (1997) 38, 1389-1396 (rice)), Weig and Komor (J. Plant Physiol. (1996) 147, 685-690 (ricinus)), Weber et al., (The Plant Cell (1997) 9, 895-908 (Vicia faba)), Shakya and Sturm (Plant Physiol. (1998) 118, 1473-1480 (carrot)), Tegeder et al. (The Plant Journal (1999) Plant J. (1999 18, 151-161 (Pisum sativum)), Noiraud et al. (Poster abstract 11^{th} Inter. Workshop on Plant Membrane Biology, Cambridge, UK (1998) (Apium graveolens)), Picaud et al., (Poster abstract 11^{th} Inter. Workshop on Plant Membrane Biology, Cambridge, UK (1998) (Vitis vinifera)) and sugar beet (access number: X83850) that with regard to the nucleotide sequences, the amino acid sequence of SUT, and their recovery, are fully incorporated in the disclosed content of the present teachings and for which protection is also being sought in conjunction with the present teachings. SUT 1-coding nucleotide sequences used in accordance with the invention as well as the amino acid sequence derived from them, represented in SEQ ID nos. 22 and 23, are also included in the subject matter of the present invention. SUT1 represents a saccharose transporter having a high affinity to saccharose but a low transport capacity for saccharose. A coexpression of saccharose transporters with differing affinities to saccharose in the same tissue—for example in sieve elements—permits the saccharose flux to be manipulated in a manner that is controlled and that is appropriate to the conditions that are actually present in the plant.

In an especially preferred embodiment of the present invention, a vector that is used to transform at least one plant cell and that contains the SUT2-coding nucleotide sequences is used. In accordance with the invention, SUT2 functions in particular as a regulator and as a sensor. SUT2 also has the biological activity of producing low-affinity saccharose transport. The transport rates of SUT2 are also low. Without being restricted by theory, SUT2 is a regulator and/or sensor of the saccharose transporter that in particular can determine its own transport activity and can also pass it on. Its transport activity can be viewed as a functional component of its sensor activity and, on the other hand, its sensor activity can be viewed as a functional component of its transport activity. SUT2,with its low affinity for saccharose, is in accordance with the invention a flux sensor that can possibly transport a substrate, namely saccharose, and that uses a signal cascade, or a portion thereof, that measures the transport rate. The affinity of SUT2 for saccharose is less than that of SUT4. In an especially preferred embodiment of the invention it was shown that the N-termini of proteins of the SUT/SUC gene family convey a modified affinity with respect to their substrate, in particular saccharose. Specifically, the N-termini of SUT2 but also those of SUT1, convey a modified saccharose affinity—in the case of SUT2 a lower affinity for saccharose, and in the case of SUT1 a higher affinity.

The invention therefore also relates to the use of N-termini of saccharose transporters, and respectively the nucleotide sequences that hold them, in particular plant saccharose transporters, to modify the saccharose transport or the saccharose sensing in plants, in particular to prepare modified saccharose transporters and sensors having modified affinities for saccharose in plants.

The invention also relates to the use of SUT2 and/or SUT2-coding DNA sequences, in particular the SUT2 loop as a regulator and sensor of saccharose transport, in particular to regulate the SUT4 and/or SUT1 activity, for example in plants or plant cells. Moreover, the invention has revealed that SUT2 can be induced by saccharose. SUT2 regulates the relative activity of saccharose transporters that are present in the same cell type, for example in the sieve elements. SUT2 in particular regulates the activity of the saccharose transporter SUT1, which has a high saccharose affinity but low transport capacity, and the SUT4 saccharose transporter, which has a high transport capacity but low saccharose affinity. This regulation can be accomplished by controlling the expression of protein activity, for example by means of protein modification or by controlling the turnover rate of mRNA or protein, leading to an increase or decrease in activity. SUT2 is expressed in plants, in particular in large leaf veins of mature leaves, blossoms, and sink organs.

The invention therefore also relates to the aforesaid N-termini and central loops, respectively loops of proteins from the SUT/SUC gene family, in particular of SUT 1, SUT2, and/or SUT4, as well as the nucleotide sequences that code these areas. These sequences are represented in a preferred embodiment in SEQ ID nos. 24, 25, and 26. The N-termini of LeSUT2 (Lycopersicon esculentum) and StSUT2 (Solanum tuberosum) comprise the first 62 amino acids of the protein and are coded by nucleotides 1 to 186 of SEQ ID no. 4 (Lycopersicon esculentum) and, respectively, no. 29 (Solanum tuberosum). The central loop of LeSUT2 is coded by nucleotides 844 to 1131 of SEQ ID no. 4; and StSUT2 is coded by nucleotides 847 to 1134 of SEQ ID no. 29.

In an especially preferred embodiment, the SUT 1-, SUT4-, and/or SUT2-coding sequences preferably located in a vector are located in a sense or antisense orientation relative to at least one regulatory element, in particular a promotor, and, for example, depending on the desired tissue specificity of one of the aforesaid promotors, are transformed in plant cells, where, depending on the integration in the genome and the expression of the product, the activity of cotransformed and/or endogenously present SUT4 is modified.

In an especially preferred embodiment of the present invention, this relates to an aforesaid process, in which a vector is transformed into the plant cell, and the vector contains SUT2-coding nucleotide sequences, preferably in the sense or antisense orientation under the operational control of a regulatory element, in particular a promotor. The vector containing the SUT2-coding nucleotide sequences may be transformed without additional vectors that, for example, contain the SUT4- or SUT1-coding nucleotide sequences. The transformation, integration, and expression of the SUT2-coding nucleotide sequences leads, on the basis of the teachings of the invention, to the SUT2 in particular being a saccharose concentration sensor and regulator having the aforesaid transporter characteristics as well as a saccharose flux sensor and regulator, and to a modification of the activity of this saccharose transport in the transformed plant, in particular of the endogenously present saccharose transporter, namely SUT4 and/or SUT 1. This regulation can occur on a transcriptional or post-transcriptional level, for example through direct protein interaction or indirectly via signal transduction.

Of course, the invention also relates to the use of the SUT2-coding nucleotide sequences or fragments thereof, in particular of the nucleotide sequence that codes the N-terminal protein area, for the transformation of plant cells, whereby said cells can be transformed together with SUT1 and/or SUT4-coding nucleotide sequences. In this case too, the overexpression, cosuppression or antisense repression of SUT2 can modify the activity of SUT 1 and/or SUT4—in other words increase or reduce them. In an especially preferred embodiment of the present invention, parts of the SUT2-coding nucleotide sequences, in particular the nucleotide sequences of SUT2 (SEQ ID no. 24) that code the N-terminal protein area or the nucleotide sequences that code the central, cytoplasmatic loop (SEQ ID no. 26) can be used in the context of chimeric gene constructs that code proteins with the biological activity of a saccharose transporter and that have, for example as an N-terminus, the SUT2-coding nucleotide sequences, for example, in the central region and at the C-terminal end, nucleotide sequences of a different saccharose transporter, for example SUT1 or SUT4. Such SUT2 nucleotide sequences that contain SUT1 or parts thereof are also identified in the present invention as modified SUT2 nucleotide sequences.

As a regulator, SUT2 interacts with other proteins, in particular with regulators, signal transduction factors, and other saccharose transporters. Thus, when SUT2 is used, additional regulators may be identified through interaction cloning. Protection is also requested for these additional regulators.

The invention also relates to preferably isolated and purified regulator proteins and sensor proteins as well as nucleotide sequences that code said proteins, that contain the central cytoplasmatic loop of SUT2, in particular chimeric proteins and nucleic acids having N- and C-terminal regions from other saccharose transporters, respectively the nucleotide sequences that code same, where said chimeric proteins and nucleic acids, respectively, contain the central loop of SUT2. The central cytoplasmatic loop has a biological activity as a regulator element and/or sensor and/or signal transducer.

In a preferred embodiment, the invention therefore relates to the aforesaid process to modify the activity of a saccharose transporter having a low affinity to saccharose but a high transport activity for saccharose relative to its known or modified SUT4-, SUT1-, and/or SUT2-coding nucleotide sequences in order to achieve the modification and to produce an improved transgenic plant.

Thus, in a preferred embodiment, the invention also relates to processes for preparing transgenic, modified plants, that have a modified activity of the said saccharose transporter and, preferably integrated in a stable manner in the genome, contain modified SUT1, SUT2 and/or SUT4 nucleotide sequences. The invention also relates to transgenic plants, plant cells, organs, or portions of organs and plants produced in this manner that are characterized by the modified activity of the said saccharose transporter and contain at least one of the said nucleotide sequences selected from the group comprising the nucleotide sequences, in particular genes for saccharide transporters such as SUT and SUC genes, preferably for SUT1; SUT2; SUT4; SUT1 and SUT2; SUT1 and SUT4; SUT2 and SUT4; SUT1 and SUT2, and SUT4. In conjunction with the present invention a modified nucleotide sequence is understood to mean a nucleotide sequence that deviates from the wild-type sequence, in particular the wild-type gene, for example a deviation due to nucleotide insertions, inversions, deletions, replacements, additions, or similar processes. For example, the modified nucleotide sequences also represent those genes that contain the coding nucleotide sequence from the wild-type, where said coding nucleotide sequence is operationally linked in the sense or antisense orientation with a heterologous promotor, for example a tissue-specific or constitutive expression promoter. In conjunction with the present invention, a modified nucleotide sequence may also be present if it corresponds exactly to the wild-type sequence. However, it is present as a naturally occurring sequence, although with an additional number of copies and/or at a different site in the genome.

A modified nucleotide sequence is also present when the nucleotide sequence that naturally occurs in endogenous form was changed by means of mutagenesis, for example transposon mutagenesis. In conjunction with the present invention, modified genes are understood to mean those nucleotide sequences that in the nucleotide sequence of their regulatory and/or protein-coding areas contain deviations, for example inserts, additions, deletions, replacements, etc. relative to the wild-type sequence, and that can therefore be referred to as mutants, derivatives, or functional equivalents. Modified nucleotide sequences and modified genes respectively can also be chimeric nucleotide sequences or genes, for example such protein-coding areas comprised of two or more nucleotide sequences that do not occur together naturally, for example constructs that have SUT2-coding sequences (SEQ ID no. 24) as the N-terminal nucleotide sequence, and that have SUT1-coding sequences as the central and 3′-terminal area. In accordance with the invention, modified genes are understood to mean those that, as a 5′-coded area, contain sequences of the SUT1 gene (for example: SEQ ID no. 25) and as a medium range and/or 3′-area contain sequences of SUT2 gene.

Modified genes can therefore contain, for example, the wild-type coding sequences and heterologous promoters, for example from other organisms or from other genes.

In the context of the present invention, a gene is understood to mean a protein-coding nucleotide sequence that is under the operative control of at least one regulatory element.

The invention also relates to means for modifying the saccharose transport. These means are nucleic acid molecules, coding a saccharide transporter having low saccharide affinity and high transport capacity for the saccharide, or portions thereof, in particular saccharose, selected from the group comprising:

a) nucleic acid molecules, comprising the nucleotide sequence shown in SEQ ID nos. 1, 2, or 27, a portion thereof, or a complementary strand thereof,

b) nucleic acid molecules that code a protein having the amino acid sequence shown in SEQ ID nos. 5, 6, or 28, and

c) nucleic acid molecules that hybridize with one of the nucleic acid molecules cited under a) and b).

In an especially preferred embodiment, the saccharide transporter is a saccharose transporter, in particular SUT4,for example from arabidopsis (Arabidopsis thaliana, At), tomato (Lycopersicon esculentum, Le), or potato (Solanum tuberosum, St). The aforesaid nucleic acid molecules are also characterized as SUT4-coding sequences.

The invention also relates to nucleic acid molecules coding a sensor and/or regulator for the saccharose transport in plants and having the properties of a low-affinity saccharose transporter with low transport rates, or portions thereof selected from the group comprising

a) nucleic acid molecules comprising the nucleotide sequence shown in SEQ ID nos. 3, 4, 24, 26, or 29, a portion thereof, or a complementary strand thereof,

b) nucleic acid molecules that code a protein having the amino acid sequence shown in SEQ ID nos. 7, 8, or 30, and

c) nucleic acid molecules that hybridize with one of the nucleic acid molecules enumerated under a) and b).

In an especially preferred embodiment the saccharide sensor and/or saccharide regulator is a saccharose sensor and/or regulator, in particular SUT2,for example from potato, tomato, or arabidopsis plants. The aforesaid nucleic acid molecules are also referred to as SUT2-coding sequences.

The nucleic acid molecules of the invention, or those that are used in accordance with the invention, may be isolated and purified from natural sources, for example from the potato plant, or they can be synthesized using known methods. Known molecular biological techniques can be used to insert various mutations in the nucleic acid molecules of the invention or into the already known nucleic acid molecules that are used in accordance with the invention resulting in the synthesis of proteins that may have modified biological properties and that may also be included in the subject matter of the invention. Mutations in accordance with the inventions also relate to all deletion mutations leading to shortened proteins. For example, modifications of the activity and the regulation of the protein can be accomplished by other molecular mechanisms such as insertions, duplications, transpositions, gene fusion, nucleotide exchange, or also through gene transfer between different strains of microorganisms and other means. In this way, mutant proteins can be produced that, for example, have a different transport capacity or a different saccharose affinity and/or that are no longer subject to the regulation mechanisms that are normally present in the cells or are subject to said mechanisms in a different form. In addition, mutant proteins in accordance with the invention can be prepared that have a modified stability, substrate-specificity, or a modified effector pattern (or a modified activity, temperature, pH, and/or concentration profile). Furthermore the teachings of the inventions apply to proteins that have a modified active protein concentration, pre- and post translational modifications, for example signal and/or transport peptides, and/or other functional groups.

The invention also relates to nucleic acid molecules that hybridize with the aforesaid nucleic acid molecules of the invention. In the context of the invention, hybridization means a hybridization under conventional hybridization conditions such as those described in Sambrook et al. (Molecular Cloning. A Laboratory Manual, Cold Spring Harbor Laboratory Press, 2nd ed., 1989), preferably under stringent conditions. In the present invention, the term hybridization is used if a positive hybridization signal is observed after washing for 15 minutes with 2×SSC and 0.1% SDS at 52° C., preferably at 60° C., and more preferably at 65° C., preferably for 15 minutes 0.5×SSC and 0.1% SDS at 52° C., preferably at 60° C. and more preferably at 65° C. A nucleotide sequence that hybridizes under such washing conditions with one of the nucleotide sequences stated in the sequence protocols is a nucleotide sequence of the invention.

The identification and isolation of such nucleic acid molecules can be performed using the nucleic acid molecules of the invention or portions of these molecules, or a complementary strand. Nucleic acid molecules that have precisely the nucleotide sequences shown in SEQ ID nos. 1, 2, 3, or 4 or that essentially correspond to these sequences, or that have portions of these sequences can be used, for example, as the hybridization sample. When fragments are used as the hybridization sample, such fragments may be synthetic fragments prepared with the aid of customary synthesis techniques whose sequence essentially corresponds to that of a nucleic acid molecule of the invention. The molecules hybridized with the nucleic acid molecules of the invention also comprise fragments, derivatives, and allelic variants of the nucleic acid molecules described above that code a protein of the invention. The term “fragments” is understood to mean portions of the nucleic acid molecules that are long enough to code the described protein.

The expression “derivative” when used in conjunction with the invention means that the sequences of the molecules differ from the sequences from the described nucleic acid molecules at one or more positions, but that they have a high degree of homology with these sequences. Homology means a sequence identity of at least 70%, preferably an identity of at least 75%, more preferably over 80% and even more preferably over 90%, 95%, 97%, or 99% at the nucleic acid level. The proteins coded by these nucleic acid molecules have a sequence identity with the amino acid sequence given in SEQ ID nos. 5, 6, 7, or 8 of at least 80%, preferably 85% and more preferably of over 90%, 95%, 97%, and 99% on the amino acid level. The deviations from the nucleic acid molecules described above may result, for example, from deletion, substitution, insertion, or recombination. These variations may be naturally occurring, for example sequences from other organisms or mutations, whereby said mutation may occur through natural means, or through systematic mutagenesis (UV or X-ray radiation, chemical agents, or other). In addition, the variations may involve synthetically produced sequences. The allelic variants may be naturally occurring variants as well as synthetically prepared variants or variants produced by means of recombinant DNA techniques. The proteins coded by the various variants of the nucleic acid molecules of the invention have certain shared characteristics such as activity, active protein concentration, posttranslational modifications, functional groups, molecular weight, immunological reactivity, confirmation, and/or physical properties such as movement behavior in gel electrophoresis, chromatographic behavior, sedimentation coefficients, solubility, spectroscopic properties, stability, optimum pH, isoelectric pH, optimum temperature, and/or others.

The nucleic acid molecules of the invention may be DNA and RNA molecules. DNA molecules of the invention are, for example, genomic DNA or cDNA molecules.

The invention also relates to vectors that contain the nucleic acid molecules of the invention.

In conjunction with the invention, the vectors may be, for example, plasmids, liposomes, cosmids, viruses, bacteriophages, shuttle vectors, and other vectors commonly used in gene technology. The vectors can have additional functional units that cause or contribute to a stabilization and/or replication of the vector in a host organism.

A preferred embodiment of the invention also includes vectors in which the nucleic acid molecule contained in said vectors is operatively attached to at least one regulatory element that produces the transcription and synthesis of translatable nucleic acid molecules in procaryotic and/or eucaryotic cells. Such regulatory elements may be promoters, enhancers, operators, and/or transcription termination signals. Needless to say, the vectors may also contain antibiotic resistance genes, herbicide resistance genes, thus, for example, selection markers.

In a preferred embodiment the invention also relates to the aforesaid vectors in which said vectors contain, in addition to nucleic acid sequences that are under the control of at least one regulatory element and that code the SUT4 and/or SUT2 in accordance with the invention, a nucleic acid sequence that codes SUT1 and that is also under the control of at least one regulatory element. Such a vector therefore has the genetic information for at least two proteins involved in the transporter saccharose. Such vectors allow the system of saccharose transport in a plant to be easily modified in a controlled and comprehensive way.

The invention also relates to host cells that integrate one of the nucleic acid molecules of the invention or one of the vectors of the invention in a stable manner or contain said molecules or vectors in a transient manner or are transformed with them and preferably are able to express SUT4 and optionally SUT1 and/or SUT2. The invention also relates to host cells that descend from a host cell that has been transformed with the nucleic acid molecules of the invention or with the vectors of the invention. The invention therefore relates to host cells that contain the nucleic acid molecules of the invention or the vectors of the invention, where a host cell is understood to mean an organism that is able in vitro to take in recombinant nucleic acid molecules and, optionally, to synthesize proteins coded by the nucleic acid molecules of the invention. Preferably, these cells are procaryotic or eucaryotic. Above all, the invention relates to microorganisms that contain the vectors, derivatives, or portions of vectors of the invention and that permit said vectors, derivatives or portions of vectors to synthesize proteins having a saccharose transport activity. The host cell of the invention can also be characterized by the fact that the nucleic acid molecule that is introduced in accordance with the invention is either heterologous with respect to the transformed cell—which means that it does not occur naturally in the cell—or that it is located at a different site or a different copy number in the genome than the corresponding naturally occurring sequence.

In one embodiment of the invention, this host cell is therefore a procaryotic cell, preferably a gram-negative procaryotic cell, more preferably an enterobacteria cell. The transformation of procaryotic cells with exogenous nucleic acid sequences is familiar to a person skilled in the art of molecular biology.

In a further embodiment of the invention, however, the cell of the invention may also be a eucaryotic cell, such as a plant cell, a fungus cell, for example yeast, or an animal cell. Processes to transform, and respectively transfect, eucaryotic cells with exogenous nucleic acid sequences are familiar to a person skilled in the art of molecular biology.

The invention also relates to cell cultures or callus tissue that have at least one of the host cells of the invention, where the cell culture of the invention or the callus in particular is able to produce a protein having a saccharose transport activity.

In one embodiment of the invention, the nucleotide sequence used in accordance with the invention is linked in the vector to a nucleic acid molecule that codes a functional signal sequence for transporting the protein to different cell compartments or to the plasma membrane. This modification can consist, for example, of an addition of an N-terminal sequence from a higher-level plant, but other modifications that cause a sequence to fuse with the coded protein are also included in the subject matter of the invention.

The expression of the nucleic acid molecule of the invention in procaryotic cells, for example in Escherichia coli, or in eucaryotic cells, for example in yeast, is interesting in that it is possible in this way, for example, to characterize the activity of the proteins that are coded by this molecule in a more precise manner.

A further embodiment of the invention comprises preferably purified and isolated peptides or proteins, coded by the nucleotide sequences of the invention, preferably with the amino acid sequences of SEQ ID nos. 5 to 8, 23 to 26, 28, or 30, preferably having the activity of a saccharose transporter, preferably a saccharose transporter having a low affinity to saccharose and a high transport capacity for saccharose, and, respectively, having the activity of a sensor or regulator of the transport of saccharose as well as processes for their preparation, in which a host cell of the invention is cultivated under conditions that permit the protein to be synthesized and then the protein is isolated from the cultivated cells and/or the culture medium.

The invention further relates to the monoclonal or polyclonal antibodies that specifically react with these proteins.

By preparing the nucleic acid molecules of the invention, it is possible, with the aid of genetic engineering methods to modify the saccharose transport in tissues of any given plant in a way that was not possible with conventional methods in plants—for example by means of breeding—and to modify the transport in such a way that it can be used to selectively change the saccharose concentration in certain tissues of a plant. By increasing the activity of the proteins of the invention, for example by means of the overexpression of appropriate nucleic acid molecules, or by providing mutants that are no longer controlled by the cell's own regulatory mechanisms and/or that have different temperature dependencies relative to their activities, it is possible to increase yields in plants that have been modified in this manner through genetic engineering. Thus, the nucleic acid molecules used in accordance with the invention can be expressed in plant cells in order to increase the activity of the corresponding saccharose transporters, or it is possible to express them in cells that normally do not express this protein. Moreover, it is possible to modify the nucleic acid molecules used in accordance with the invention using methods that are known to a person skilled in the art in order to obtain proteins of the invention that are no longer subject to the cell's own regulatory mechanisms or that have modified temperature dependencies or substrate/product specificities. The invention also allows the synthesized protein to be localized in any given compartment or in the plasma membrane of the plant cell. In order to achieve localization in a specific compartment or in the plasma membrane, the coded region may need to be linked with DNA sequences that accomplish the localization in the respective compartment or plasma membrane. Such sequences are known (see, for example, Braun et al., EMBO J. (1992) 11, 3219-3227; Wolter et al., Proc. Natl. Acad. Sci. USA (1988) 85, 846-850; Sonnewald et al., Plant S. (1991) 1, 95-106).

The invention therefore also relates to transgenic plant cells that were transformed with one or more nucleic acid molecule(s) of the invention or nucleic acid molecule(s) used in accordance with the invention, as well as transgenic plant cells that descend from such transformed cells. Such cells contain one or more of the nucleic acid molecule(s) of the invention or of the nucleic acid molecule(s) used in accordance with the invention, whereby said molecules(s) is/are preferably linked to regulatory DNA elements that produce the transcription in plant cells, in particular with a promotor. The invention also relates to transgenic plant cells whose genome contains at least two stably integrated modified genes from the family comprising the SUT and/or the SUC genes. Such cells differ from naturally occurring plant cells in that they contain at least one nucleic acid molecule of the invention or nucleic acid molecule used in accordance with the invention that does not naturally occur in said cells, or in that said molecule is integrated at a site in the genome of the cell at which it does not normally occur in nature, in other words in a different genomic environment or in a different copy number than the one that normally occurs in nature. The transgenic plant cells can be regenerated to produce whole plants using techniques that are familiar to a person skilled in the art. The plants obtained through regeneration of the transgenic plant cells of the invention are also included in the scope of the present invention. The invention also relates to plants that contain at least one cell, preferably however a plurality of cells, that contain the vector systems or derivatives or fragments thereof or the vector systems or derivatives or fragments thereof used in accordance with the invention, and, based on the inclusion of said vector systems, derivatives or portions of the vector systems are capable of synthesizing proteins that cause a modified saccharose transport activity, in particular an SUT4 activity. The invention therefore allows plants of various types, genera, families, orders, and classes to be produced that have the aforesaid characteristics. The transgenic plants may, in theory, be plants of any given plant species—in other words, monocots as well as dicots, such as graminea, pinidae, magnoliidae, ranunculidae, caryophyllidae, rosidae, asteridae, aridae, liliidae, arecidae and commellinindae as well as gymnosperms, algae, mosses, ferns, or also calli, plant cell cultures, etc., as well as fragments, organs, tissue, harvest or reproductive materials thereof. Preferably the plants are agricultural plants, in particular starch-synthesizing or starch-storing plants such as wheat, barley, rice, corn, topinambur, sugar beet, sugar cane, or potatoes. However, the invention also relates to other plants such as tomatoes, arabidopsis, peas, rapeseed, sunflower, tobacco, rye, oats, manioc, lettuce, spinach, grapes, apples, coffee, tea, bananas, coconuts, palms, beans, pines, poplar, eucalyptus, etc. The invention also relates to reproductive material and harvest products from the plants of the invention, and particular blossoms, fruits, seeds, tubers, roots, leafs, taproots, sprouts, shoots, etc.

In order to express the nucleic acid molecules of the invention or the nucleic acid molecules used in accordance with the invention in the sense or antisense orientation, for example in plant cells, said molecules are linked to regulatory DNA elements that accomplish the transcription in plant cells. These elements include, in particular, promoters. In general, any promotor that is active in plants may be used for expressing the SUT1-, SUT2-, and/or SUT4-coding nucleotide sequences, for example a promotor that expresses constitutively, or that only expresses in a certain tissue, at a certain time in the development of the plant, or at a time that is determined by external factors. With regard to the plant, the promotor may be homologous or heterologous. Among the promotors that may be used are, for example, the promotor of the 35S RNA of cauliflower mosaic virus (CaMV) and the ubiquitin promotor from corn for a constitutive expression; especially preferred is the patatin gene promotor B33 (Rocha-Sosa et al., op. cit.) for a tuber-specific expression in potatoes, or a promotor that ensures that expression only occurs in tissues that are active in photosynthesis, for example the ST-LS1-promotor (Stockhaus et al., Proc. Natl. Acad. Sci. USA (1987) 84, 7943-7947,Stockhaus et al., EMBO J. (1989) 8, 2445-2451) or, for an endosperm-specific expression, the HMG promotor from wheat, the USP promotor, the phaseolin promotor, or promoters of zein genes from corn. A termination sequence may also be present in the vector. This sequence is used to correctly terminate the transcription. A poly-A tail can be added to the transcript to stabilize it. Such elements are described in the literature (Gielen et al., EMBO J. (1989) 8, 23 -29) and are fully interchangeable. Additional promotors are described above.

A large number of cloning vectors are available for inserting exogenous genes into higher-level plants. These vectors contain a replication signal for E. coli and a marker gene for selecting transformed bacteria cells. Examples of such vectors are pBR322, pUC series, M13mp series, pACYC181,etc. The desired sequence can be introduced at an appropriate restriction cut site in the vector. The resulting plasmid is used for the transformation of, for example, E. coli cells. Transformed E. coli cells are cultivated in a suitable medium, then harvested and lysed. The plasmid is recovered. The analytical methods that are generally used to characterize the plasmid DNA that is obtained are restriction analysis, gel electrophoreses, and other biochemical/molecular-biological methods. After each manipulation, the plasmid DNA can be lysed and the resulting DNA fragments can be combined with other DNA sequences. Each plasmid DNA sequence can be cloned in the same or different plasmids. A variety of techniques are available for introducing the DNA into a plant host cell. These techniques include the transformation of plant cells with T-DNA using Agrobacterium tumefaciens or Agrobacterium rhizogenes as the transformation vectors, the fusion of protoplasts, the injection and electroporation of DNA, and the incorporation of DNA using the biolistic method as well as additional methods. In the case of the injection and electroporation of DNA in plant cells, basically no specific requirements apply to the plasmids that are used. Simple plasmids such as pUC derivatives can be used. However, if entire plants are to be regenerated from cells transformed in this manner, a selectable marker should be present.

Depending on the method used to insert the SUT1-, SUT2-, and/or SUT4-coding nucleotide sequences into the plant cell, additional DNA sequences may be necessary. If, for example, the Ti- or Ri-plasmid is used for the transformation of the plant cells, at least the right border sequence but frequently also the right and left border sequence of the Ti- and Ri-plasmid-T-DNA must be attached as a lateral region to the genes that are to be introduced. If agrobacteria are used for the transformation, the DNA that is to be introduced must be cloned into specific plasmids, either into an intermediary vector or into a binary vector. On the basis of the sequences, which are homologous to sequences in the T-DNA, the intermediary vectors can be integrated by means of homologous recombination into the Ti- or Ri-plasmid of the agrobacteria. This plasmid also contains the vir region that is necessary for the transfer of the T-DNA. Intermediary vectors cannot replicated in agrobacteria. A helper plasmid can be used to transfer the intermediary vector to Agrobacterium tumefaciens. Binary vectors can replicate in E. coli as well as in agrobacteria. They contain a selection marker gene and a linker or polylinker, that are framed by the right and left T-DNA border region. They can be transformed directly into the agrobacteria (Holsters et al., Mol. Gen. Genet. (1978) 163, 181-187). The agrobacterium that serves as the host cell should contain a plasmid that has a vir region. Additional T-DNA may be present. The agrobacterium transformed in this way is used to transform plant cells. The use of T-DNA for the transformation of plant cells is described in EP-A-120 516; Hoekema: The Binary Plant Vector System, Offsetdrukkerij Kanters. B. V., Alblasserdam (1985), Chapter V, Fraley et al., Crit. Rev. Plant. Sci., 4, 1-46,and An et al. EMBO J. (1985) 4, 277-287. Plant explants may be co-cultivated with Agrobacterium tumefaciens or Agrobacterium rhizogenes to transfer the DNA into the plant cell. In a suitable medium that contains antibiotics or biocides to select transformed cells, whole plants can be regenerated again from the infected plant material, for example pieces of leafs, stem segments, roots, and also protoplasts or suspension-cultivated plant cells. The plants obtained in this manner can then be analyzed for the presence of the introduced DNA. Other methods for introducing exogenous DNA using the biolistic method or protoplast transformation are known (Willmitzer, L., 1993 Transgenic plants, In: Biotechnology, A Multi-Volume Comprehensive Treatise (H. J. Rehm, G. Reed, A. Pühler, P. Stadler, eds.), vol. 2, 627-659, VCH Weinheim: New York, Basel, Cambridge). Alternative systems to transform monocots are the electrically or chemically induced uptake of DNA in protoplasts, the electroporation of partially permeabilized cells, the macroinjection of DNA in inflorescences, the microinjection of DNA in microspores and pro-embryos, the uptake of DNA by germinating pollen and the uptake of DNA in embryos by means of swelling (Potrykus Physiol. Plant (1990), 269-273). While the transformation of dicots using Ti-plasmid vector systems with the aid of Agrobacterium tumefaciens is an established technique, recent work suggests that monocots can also be transformed using agrobacterium-based vectors. (Chan et al., Plant Mol. Biol. (1993) 22, 491-506; Hiei et al., Plant J. (1994) 6, 271-282; Bytebier et al., Proc. Natl. Acad. Sci., USA (1987) 84, 5345-5349; Raineri et al., Bio/Technology (1990) 8, 33-38; Gould et al., Plant. Physiol. (1991) 95, 426-434; Mooney et al.; Plant, Cell Tiss. & Org. Cult. (1991) 25, 209-218; Li et al., Plant Mol. Biol. (1992) 20, 1037-1048). Some of the cited transformation systems have been established for various grains: the electroporation of tissues, the transformation of protoplasts, and the transfer of DNA through bombardment of particles into regenerable tissue and cells (Jähne et al.; Euphytica 85 (1995), 35-44). The transformation of wheat is described in the literature (Maheshwari et al., Critical Reviews in Plant Science (1995) 14(2), 149-178) and of corn described in Brettschneider et al. (Theor. Appl. Genet. (1997) 94, 737-748), and Ishida et al. (Nature Biotechnology (1996) 14, 745-750).

The invention also relates to identification and/or screening processes for modulators of saccharose metabolism, preferably potential pesticides and herbicides, in which SUT4- and/or SUT2-expressing cells or tissue, in particular host cells or plants of the invention, for example yeast or plant cells of the invention, are brought into contact with the potential modulator that is to be tested, for example the pesticide or herbicide, and the effect, in particular the inhibitory effect of the potential modulator, for example of the pesticide or herbicide, on the activity of SUT1, SUT2, and/or SUT4 is determined quantitatively or qualitatively. Likewise, SUT2 and/or SUT4 may be used to develop systems that permit the improved mobilization of pesticides. Inhibitors may be identified by systematically screening chemical libraries for substances that specifically block the growth of yeasts, that express low-affinity saccharose transporters such as SUT4, or that co-express the combinations of other saccharose transporters, for example SUT4 and SUT2 or SUT2 and SUT1 or SUT4 and SUT1. These inhibitors could be used as herbicides or also as precursors to new herbicides. Based on the tests, potential pesticides that can be mobilized in the plant by means of these transporters and in this way can reach their target location more effectively can also be identified.

The invention also relates to influencing chimeraplasty in other words influencing the activity of the transporters in the plant through the use of mixed oligonucleotides, which either increases the activity of the saccharose transporters, lowers it, or modifies the biochemical properties of the saccharose transporters. A process of this type is described in WO99/07865, which, with regard to this process, is fully included in the disclosed contents of the present teachings and is being claimed in the scope this invention.

The invention also relates to the use of SUT2-coding nucleotide sequences or of SUT2 to identify modulators, in particular inductors, activators, or inhibitors of the saccharose transport, in particular the sensing and/or regulation of the saccharose transport in a plant, whereby the activity of the protein that is coded by the SUT2-coding nucleotide sequences is detected in the presence and absence of a potential modulator. The activity of a saccharose transporter that is regulated by SUT2, for example SUT1 or SUT4, can be identified instead of the activity of SUT 2.

The invention also relates to the use of SUT 1-coding nucleotide sequences, in particular the SEQ ID no. 22 and/or of SUT 4-coding nucleotide sequences and/or of SUT 1 and/or SUT 4 to identify modulators of the saccharose transport activities in a plant, in particular of an inhibitor of low-affinity of the high-capacity loading of the phloem with saccharose, whereby the activity of a protein coded by the SUT4-nucleotide sequences is detected in the presence and in the absence of the potential modulator.

The invention also relates to the use of the SUT1, SUT2, and SUT4 nucleotide sequences of the invention to identify homologous genes in other plants, for example plants from cDNA or genomic banks.

The invention also relates to the use of the genes and the surrounding regions as molecular markers for crossing programs. Both SUT2 and SUT4 loci are located in the regions of QTL loci for higher carbohydrate content and higher yields in potato tubers. Therefore, both are suitable for use in breeding programs involving wild types or high-performance types for crossing in suitable chromosome fragments and in this way obtaining plants that produce improved yields.

Additional preferred embodiments of the invention are recited in the dependent claims.

The sequence protocol contains:

SEQ ID no. 1; the coding DNA sequence of the SUT4 gene from Arabidopsis thaliana.

SEQ ID no. 2; the coding DNA sequence of the SUT4 gene from Lycopersicon esculentum

SEQ ID no. 3; the coding DNA sequence of the SUT2 gene from Arabidopsis thaliana.

SEQ ID no. 4; the coding DNA sequence of the SUT2 gene from Lycopersicon esculentum.

SEQ ID no. 5: the amino acid sequence of SUT4 from Arabidopsis thaliana.

SEQ ID no. 6: the amino acid sequence of SUT4 from Lycopersicon esculentum.

SEQ ID no. 7: the amino acid sequence of SUT2 from Arabidopsis thaliana.

SEQ ID no. 8: the amino acid sequence of SUT2 from Lycopersicon esculentum.

SEQ ID no. 9: a T-DNA-specific primer.

SEQ ID no. 10: an SUT4-specific primer.

SEQ ID no. 11: an SUT4-specific primer.

SEQ ID nos. 12 to 15 represent additional SUT4 primers.

SEQ ID nos. 16 and 17 represent the amino acid sequence of sections of LeSUT4.

SEQ ID no. 18 to 21 represent cloning primers.

SEQ ID no. 22: the coding DNA sequence of the SUT1 gene from Solanum tuberosum.

SEQ ID no. 23: the amino acid sequence of SUT1 from Solanum tuberosum.

SEQ ID no. 24: the DNA sequence from SEQ ID no. 3 that codes the N-terminal region of SUT2, having the nucleotides 1 to 239.

SEQ ID no. 25: the DNA sequence for SEQ ID no. 22 that codes the N-terminal region of SUT1, having the nucleotides 1 to 149.

SEQ ID no. 26: the DNA sequence or SEQ ID no. 3 that codes the central loop having the nucleotides 843 to 1130.

SEQ ID no. 27: the coding DNA sequence of the SUT4 gene from Solanum tuberosum.

SEQ ID no. 28: the amino acid sequence of SUT4 from Solanum tuberosum.

SEQ ID no. 29: the coding DNA sequence of the SUT2 from Solanum tuberosum.

SEQ ID no. 30: the amino acid sequence of SUT2, from Solanum tuberosum.

The invention shall now be explained based on the following examples and the appurtenant figures.

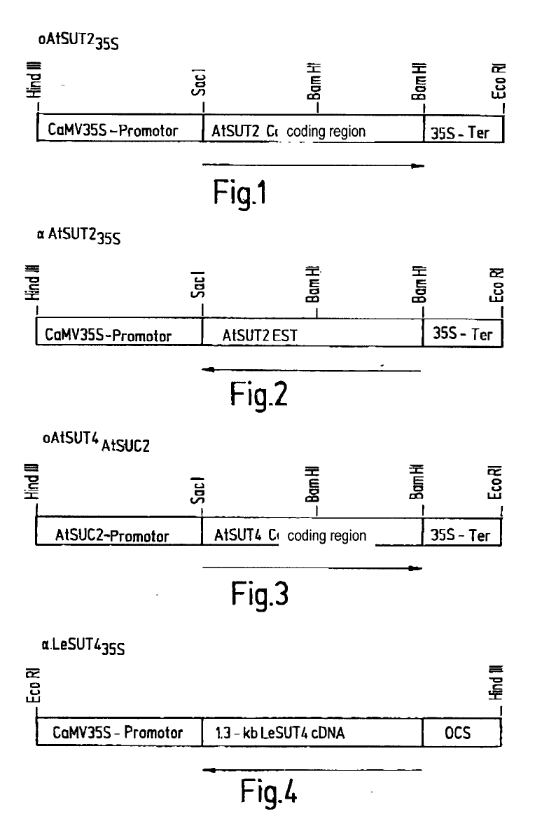

FIGS. 1 to 4 show a schematic representation of the structure of the gene construct used in accordance with the invention.

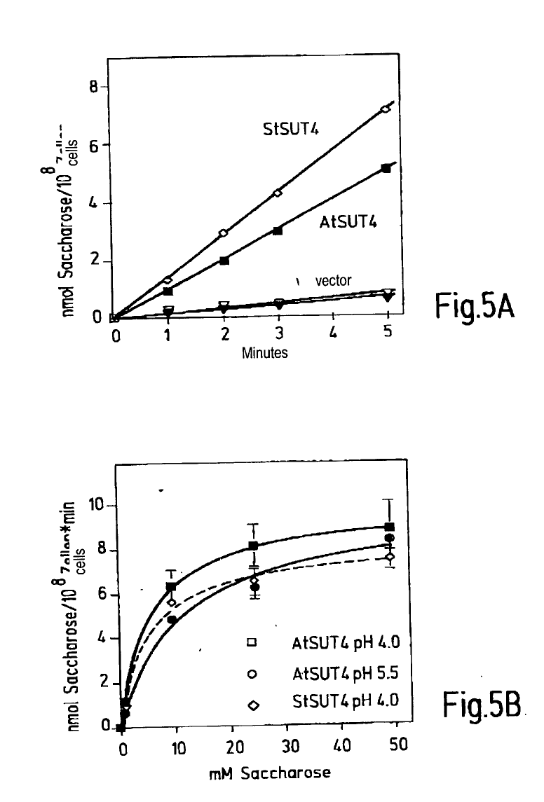

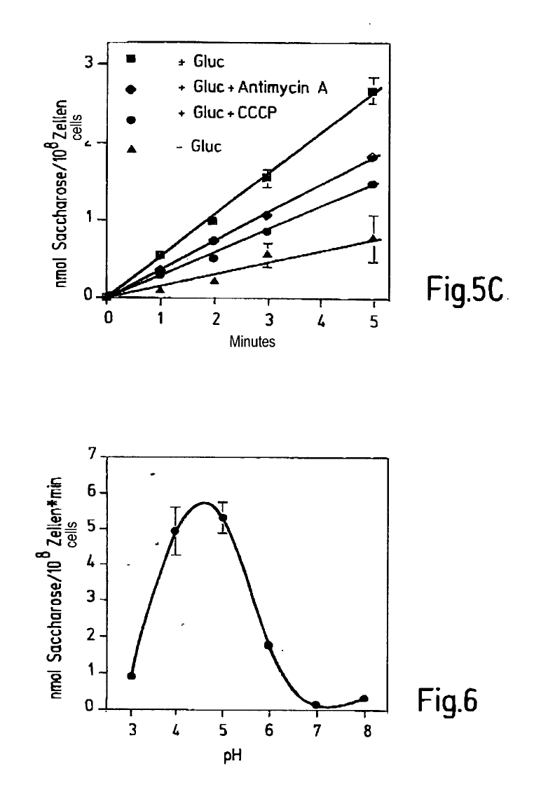

FIGS. 5 and 6 show graphic representation of the protein activities of SUT4.

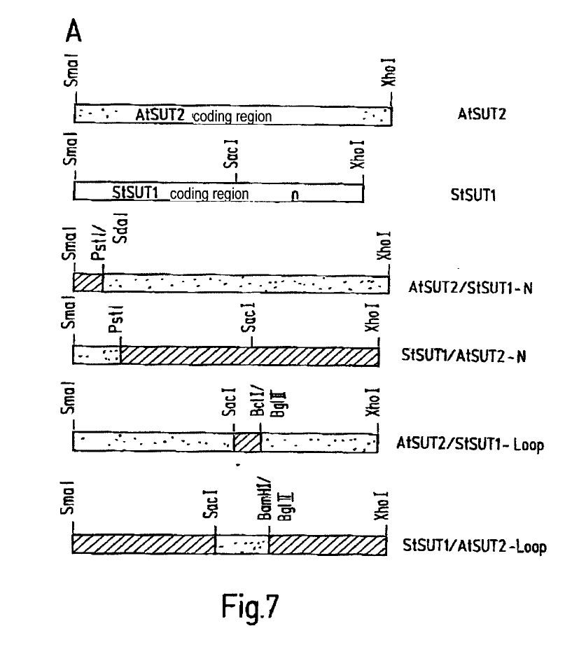

FIG. 7 shows chimeric SUT2 and SUT1 gene constructs.

### EXAMPLE 1

Isolation of SUT4 cDNA

In the database (gene bank) sequences that were not previously identified and that have a distant homology to SUT1 were identified.

Genomic Sequence: AtSUT4 AC000132

A cDNA was amplified by means of PCR from an Arabidopsis seedling bank (Minet et al., Plant J. (1992) 2, 417-422). Primers based on the genomic sequence (5′-gactctgcagcgagaaatggctacttccg SEQ ID no. 12, 5′-taacctgcaggagaatctcatgggagagg SEQ ID no. 13) were designed. Each of the primers contains one PstI restriction cut site (underlined), and they are designed in such a way that the entire coding sequence of AtSUT4 is amplified. A product having an expected size of 1566 bp was cut with PstI and ligated into the PstI site of the vector pBC SK+ (Stratagene). AtSUT4 was subcloned into the PstI site of pDR196. The AtSUT4-cDNA in pDR196 was sequenced in both directions. Ecotype differences between the SUT4 gene in Columbia and Landsberg erecta were verified by sequencing AtSUT4 from Landsberg erecta.

A tomato (Lycopersicon esculentum cv. UC82b) cDNA-bank (blossoms) was sampled with a 300 bp eco-RIBg1II fragment of the genomic tobacco clone NtSUT3 (Lemoine et al., FEBS Lett. (1999) 454, 325-330) with reduced stringency.

Three different clones independent of LeSUT1 were isolated and named LeSUT4. The orthologic sequence obtain by means of RT-PCR from the potato plant was isolated using primers of the LeSUT4 sequence and was named StSUT4. StSUT4 was cloned as a PstI/NotI fragment into pBC SK− (Stratagene) and subcloned as a XhoI/SacII fragment into the yeast expression vector pDR195, which has a URA3 marker, PMA1 promotor, and ADH1 terminator (Rensch et al., FEBS Lett. (1995) 370, 264-268). The following were used to amplify StSUT4 (ORF,=cDNA sequence): 5′-SUT4-PstI 5′-GAGACTGCAGATGCCGGAGATAGAAAGGC-3′ (SEQ ID no. 14) 5′-SUT4-NotI 5′-TATGACAGCGGCCGCTCATGCAAAGATCTTGGG-3′ (SEQ ID no. 15).

### EXAMPLE 2

Isolation of SUT2 cDNA

Sequences that have a distant homology to SUT1 and that were not previously described were identified in the database (gene bank).

Genomic Sequence: AtSUT2 AC004138

Based on detailed comparison of sequences, other known homologous primer sequences that could permit cloning of the potential cDNA sequence via RT-PCR from leaf mRNA were identified.

Primers based on the genomic sequence (5′ TACGAGAATTCGATCTGTGTGTTGAGGACG, SEQ ID no. 20, 5′ AGAGGCTCGAGTGGTCAAAAAGAATCG, SEQ ID NO. 21) were designed. The primers contain an EcoRI or an XhoI restriction cut site (underlined) and are designed in such a way that the entire coding sequence of AtSUT2 is amplified. A product having the expected size of 1785 bp was cut using EcoRI and XhoI and ligated in an oriented position into the vector pDR196.

### EXAMPLE 3

Functional Analysis of SUT4

The yeast strain SUSY7/ura3 is a modified version of SUSY7 (Riesmeier et al., EMBO J. (1992) 11, 4705-4713), which contains a deletion of a portion of the URA3 gene and, as a result, permits selection for uracil auxotrophy. Media that contained the 1.7 g/L yeast nitrogen base without amino acids (Difco), 2% saccharose, 20 mg/L tryptophane, and 1.5% agarose, pH 5.0, were used to analyze yeast growth on a saccharose medium.

For the saccharose uptake tests, yeast was cultivated in a liquid minimal medium containing glucose, up to an OD₆₂₃ of approximately 0.8. Cells were collected by means of centrifugation, washed in 25 mM sodium phosphate buffer (pH 5.5), and suspended in the same buffer at an OD₆₂₃ of 20. The uptake tests were initiated by adding glucose ending at a final concentration of 10 mM to the yeast cells one minute before adding ¹⁴C-saccharose. Following incubation at 30° C. while stirring, cells were collected by means of vacuum filtration on fiberglass filters (1-5 minutes), washed twice with 4 mL 10 mM saccharose (at the freezing point), and the radioactivity was determined using a liquid scintillation counter. The AtSUT4 expression permitted yeast to grow on saccharose. AtSUT4 and StSUT4 proved to be functional saccharose transporters. The curve over time for the uptake of ¹⁴C-saccharose of AtSUT4- or StSUT4-expressing yeast is shown in FIG. 5A. A significant difference compared with the vector controls is apparent.

Data for the kinetic analysis were obtained for SUT4 from a nonlinear regression of the uptake measurements using the Michaelis-Menten equation. Kₘ was represented as an average of eight determinations using three independent transformants±standard deviation. The K_{M} value for saccharose that was determined was 11.6±0.6 mM (at a pH of 5.5) and 5.9±0.8 mM (at a pH of 4.0) for SUT4 from arabidopsis. For SUT4 from Solanum tuberosum the K_{M} value was 6.0±1.2 mM (pH 4.0) (see FIG. 5B).

The stimulation of the uptake of ¹⁴C-saccharose by means of SUT4 through glucose and the inhibition through an electron transport inhibitor (antimycin A and the protonophore CCCP is shown in FIG. 5C). FIG. 6 shows the pH optimum of the SUT4-induced saccharose transport.

### EXAMPLE 4

Preparation of an SUT4-insertion Mutant (Arabidopsis thaliana)

Seeds of 12,800 T-DNA-mutagenized arabidopsis plants (corresponding to 19,200 insertion operations) were obtained from Dupont Co. and from Arabidopsis Biological Resource Center (ABRC), Ohio State University. The plants were tested in groups of 100. The plants were allowed to grow in a sterile culture, and genomic DNA was isolated. The DNA from the 140 groups of 100 plants each was consolidated into 14 super-groups (superpool) and was screened using the method developed by Krysan et al. (Proc. Natl. Acad. Sci. USA (1996) 93, 8145-8150) whereby gene-specific and T-DNA-specific primers were used. A PCR was performed using the superpool DNA as a template, with a T-DNA-specific primer (LB, left border region SEQ ID no. 9) and a gene-specific primer (AtSUT4r2 SEQ ID no. 10). The PCR products were separated by means of agarose gel electrophoresis and transferred to a charged nylon membrane. The membrane was hybridized with a PCR product of 2.46 kb length, prepared from WS (Wassilewskija) genomic DNA as a template and the primers AtSUT4r2 (see above) and AtSUT4f2 (ATGGCTACTTCCGATCAAGATCGCCGTC SEQ ID no. 11). This probe was marked with ³²P-CTP.

A superpool was identified by hybridizing the marked probe with the blot. DNAs from the pools of 100 plants that form the superpool were then screened in the same manner: PCR was performed with DNAs from pools of 100 as the template and AtSUT4r2 and LB as primers; DNA blot hybridization was performed with the AtSUT4 genomic probe (2.46 kb) to detect amplified products.

Positive hybridization was observed in pool CS2165, which comprised 100 T-DNA-mutagenized lines. Individual plants of CS2165 were cultivated and genomic DNA was prepared. The DNA of individual plants was screened as described. PCR was performed using DNA from the individual plants as the template and AtSUT4r2 and LB as primers. PCR products were made visible on agarose gel by staining them with ethididium bromide. The PCR product was sequenced with AtSUT4r2 and LB as sequence primers. A sequence that is identical to the AtSUT4 gene indicates a T-DNA insertion in the AtSUT4 gene.

In group CS2615 (Ohio State University, ABRC) a plant was obtained that produced positive results both with a T-DNA-specific primer(LB 5′-GATGCACTCGAAATCAGCCAATTTTAGAC) (SEQ ID no. 9) as well as with an SUT4-specific primer (AtSUT4r2 5′-TCATGGGAGAGGGATGGGCTTCTGAATC) (SEQ ID no. 10). Individual plants were isolated and the insertion site of the T-DNA was sequenced, whereby it was revealed that the left border sequence of the T-DNA was present about 480 base pairs upstream from the ATG of the SUT4 gene. The mutant was crossed back two times with WS (Wassilewskija). It was found that the kanamycin resistance segregated in a ratio of 2.9:1 (427:147), which indicated the presence of a single marked locus. Homozygotes were obtained. These plants had significantly more starch than the WS wild type, which could be proved by KI (potassium iodide) staining. In addition, the plants exhibited vigorous sprout growth under light. Both results clearly show that AtSUT4 plays an important role upon the export of saccharose from source organs, namely leaves. RT-PCR shows that the mRNA of SUT4 is present in the mutant, and that the mutant therefore is not a “knockout” plant.

### EXAMPLE 5

Isolation and RNase Protection Analysis

RNA was isolated from various organs of a tomato plant cultivated in a greenhouse (L. esculentum, cv. Moneymaker) using the Schwacke method (Schwacke et al., Plant Cell (1999) 11, 377-392). Reverse transcription was performed using the MAXIscriPt™ SP6/T7 in vitro Transcription Kit (Ambion), using α-³²P UTP. A 600 bp PCR product was obtained from pSport, which contained the 340 bp LeSUT4 fragment. This fragment was used as a template. The probe could not be purified further, and 300,000 CPM were hybridized per sample. Hybridization was performed overnight with 20 μg RNA at 45° C. Following the digestion of RNA, the protected RNA was separated on a 5% polyacrylamide gel (13×15 cm) at 150 mV. The gels were dried and subjected to X-ray imaging.

In an RNA blot analysis and in the RNA protection analysis, the strongest expression of SUT4 was found in sink leaves, stems, cotyledons, and in unripe fruits. Low expression was found in source leafs.

### EXAMPLE 6

Preparation of Anti-SUT4 Antisera and Immunolocalization

Rabbits were immunized using synthetic peptides linked with KLH, corresponding either to the N-terminus (MPEIERHRTRHNRPAIREPVKPR SEQ ID no. 16) or the central loop (GSSHTGEEIDESSHGQEEAFLW SEQ ID no. 17) of LeSUT4. An affinity purification of the antisera was performed as previously described (Kühn et al., (1997) op. cit.) using synthetic peptides combined with CNBr-activated Sepharose 4B columns (Pharmacia). Pre-immune serum was purified using the same method, except that protein A Sepharose (BioRad) was used instead of peptide affinity chromatography.

Fluorescence immunodetection of SUT4 in potato and tomato plants was performed as described in the literature (Stadler et al., Plant Cell (1995) 7, 1545-1554) using the modifications described below. Hand-cut sections (1 mm) of tomato plant and potato plant stems were fixed over night under vacuum in Mops buffer (50 mM Mops/NaOH, pH 6.9, 5 mM EGTA, 2 mM MgCl₂) containing 0.1% glutaraldehyde and 6% formaldehyde. After washing three times with Mops buffer on ice, the fragments were dehydrated by means of incubation in an ethanol series, followed by two incubations in 96% ethanol. Following incubation overnight in 1:1 ethanol, methyl acrylate mixture (75% [vol./vol.] butyl methyl acrylate, 25% [vol./vol.] methyl methacrylate, 0.5% benzoin ethyl ether, 10 mM DTT), the material was embedded in 100% methyl acrylate mixture. The polymerization took place overnight under UV light (365 nm) at 4° C. Semi-thin sections (1 μm) were placed on a pre-heated Histobond slide (Camon) and dried at 50° C.

To remove the methyl acrylate from the sections, the slides were incubated for 30 seconds in acetone, rehydrated by means of an ethanol series, and blocked for 1 hour using 2% BSA in PBS (100 mM sodium phosphate, pH 7.5, 100 mM NaCl). After incubation overnight with affinity-purified antibodies to LeSUT4, the slides were washed twice in PBS-T (PBS with 0.1% Tween) and once with PBS, followed by a 1-hour incubation with anti-rabbit conjugate IgG-FITC (fluorescin isothiocyanate). After three washing steps with PBS-T, PBS and distilled water, photomicrographs were made using a fluorescence-phase microscope (Zeiss, Axiophot) and exciter light of 450-490 nm.

Fluorescence signals were only detected in sieve elements (tomato and potato).

### EXAMPLE 7

Preparation of Transgenic Plants

The AtSUT2 Overexpression Construct (oAtSUT2_{35S})

AtSUT2 cDNA was amplified by means of PCR—the product was 1,785 bp long, corresponding to the coding region from ATG (position 1) to TGA (position 1785) in the coding region of the invention. The fragment was cloned in a sense orientation into a 35S promotor expression cassette (pBinAr35S), which was isolated as an eco-RI/HindIII fragment of pBinAr (Höfgen and Willmitzer, Plant Sc. (1990) 66, 221-230). This construct was cut with HindIII and EcoRI and was cloned into the HindIII/EcoRI-cut pGTPV-bar (Becker et al., op. cit., Plant Mol. Biol. (1992) 20, 1195-1197). Plants were transformed.

The AtSUT2 Antisense Construct (αAtSUT2_{35S})

AtSUT2 cDNA (ATTS5034EST access number) was cut with SacI and BamHI and cloned in the antisense orientation into the pBinAr35S expression cassette. This construct was cut with HindIII and EcoRI and cloned into the HindIII/EcoRI-cut pGPTV-bar (Becker et al., op. cit.). Plants were transformed.

The AtSUT4 Overexpression Construct (oAtSUT4_{ATSUC2})

AtSUT4 cDNA was amplified. The 1,533 bp fragment begins by means of PCR at position 1 of AtSUT4cDNA sequence of the invention and ends at TAG position 1533. The SUC2 promotor was separated from arabidopsis (Columbia ecotype) genomic DNA using the following primers: (reverse 5′-ATGGCTGACCAGATTTGAC; SEQ ID no. 18 and forward 5′-GTTTCATATTAATTTCAC; SEQ ID no. 19) The 1.533 kb fragment was cloned in the sense orientation behind the AtSUC2 promotor (X79702). This construct was cut with HindIII and EcoRI and cloned into the HindIII/EcoRI-cut pGPTV-bar (Becker et at., op. cit.). Plants were transformed.

LeSUT4 antisense construct (αLeSUT4_{35S})

The LeSUT4 cDNA was cut with BamHI, resulting in a 1.3 kb fragment, which was smoothed and cloned into the SmaI cutting site of pBinAR (Bevan, Nucleic Acids Research (1983) 12, 8711-8721).

FIGS. 1 to 4 show the aforesaid constructs.

### EXAMPLE 8

8.1 Preparation of a Chimeric Protein Between AtSUT2 and StSUT1

The open reading frame of AtSUT2 was isolated by means of RT-PCR from Arabidopsis thaliana (Columbia ecotype) leaves and cloned into the yeast expression sector pDR196 (Barker et al., (2000) Plant Cell 12: 1153-1164). The open reading frame of StSUT1 was amplified from the StSUT1 cDNA in pDR195 (Riesmeier et al. (1993) op. cit.), and primers having the restriction cut sites for SmaI and XhoI were used. The open reading frame was ligated into the yeast expression vector pDR196.

Chimeric constructs were prepared in which the N-terminus of AtSUT2, in other words the N-terminal region of SUT2 of the invention (coded by SEQ ID no. 24), was exchanged with the corresponding N-terminal domains of StSUT1 (coded by SEQ ID no. 25), in other words the N-terminal region of SUT1 of the invention, and vice-versa, where by means of PCR restriction cut sites were produced within a preserved region of the first transmembrane domains of AtSUT2 and StSUT1. Then, PCR fragments of the N-terminal region and of the remainder of the sequence were cloned into the yeast expression vector pDR196, whereby the cut sites SmaI and PstI were used for the N-terminal areas and PstI (SdaI for AtSUT2) and XhoI for the remaining region of the open reading frame. These chimeric constructs are referred to below as AtSUT2/StSUT1-N and StSUT1/AtSUT2-N, and they are shown in FIG. 7.

Construct StSUT1/AtSUT2-N has nucleotides 1 to 239 of SEQ ID no. 3 fused to nucleotides 150 to 1548 of StSUT1, shown in SEQ ID no. 22, whereby the construct exhibits a nucleotide replacement of t to c as a result of cloning-related factors. The fusion region of the construct is shown as a sequence below, where the lower-case letters are the sequences of SUT2 and the upper-case letters are the sequences of SUT1 (top line: no replacement, lower line: with replacement):

. . . tgggcattgca/GCTCTCTT . . .
. . . tgggcactgca/GCTCTCTT . . .

AtSUT2/StSUT1-N has nucleotides 1 to 149 of StSUT1, represented in SEQ ID no. 22, fused to nucleotides 240 to 1785 of AtSUT2 shown in SEQ ID no. 3. Because of cloning-related factors, the construct has nucleotide replacements relative to the wild-type sequence 3. The fusion region is shown below. In it the upper line is the theoretically obtained construct and the actually prepared fusion region is shown in the lower line. The upper-case letters refer to the sequences of SUT1 and the lower-case letters refer to the sequences of SUT2:

. . . TGGGCTCTTCA/actttct
. . . TGGGCTCTGCA/ggtttct

Additional chimeric constructs were prepared in which the central cytoplasmatic region, in particular loop, of AtSUT2, which is represented in SEQ ID no. 26, were replaced with the smaller cytoplasmatic region, in particular loop, of StSUT1, and vice-versa. Restriction cut sites were used by means of PCR within preserved areas of the transmembrane regions VI and VIII. The N-terminal half, the cytoplasmatic loop, and the C-terminal half of the open reading frame were amplified by means of PCR using the Pfu-polymerase (Stratagene) and were cloned into the yeast expression vector by ligating the three fragments using SacI and Bc1I/Bg1II for AtSUT2 with the StSUT1 loop and SacI and BamHI/Bg1II for StSUT1 with the AtSUT2 loop. The chimeric DNA was then ligated into the yeast expression vector pDR196 using SmaI and XhoI. These chimeric constructs are referred to below as AtSUT2/StSUT1-loop and StSUT1/AtSUT2-loop, and they are shown in FIG. 7.

The construct AtSUT2/StSUT1-loop has nucleotides 1 to 842 of AtSUT2 (SEQ ID no. 3), 750 to 893 of StSUT1 (SEQ ID no. 22), and nucleotides 1131 to 1785 of AtSUT2 (SEQ ID no. 3). The upper and lower-case letters used below have the same meaning as stated above. Likewise, the upper line represents the sequence of the theoretically-obtained construct, and the lower line shows the actual sequence of the construct including the effects of factors encountered during cloning.

1. Fusion site:

. . . tgctaaagagat/CCCGGAGA . . .
. . . tgctaaagagct/CCCGGAGA . . .

2. Fusion site:

. . . GTTTGAACTG/gttatcctgg
. . . GTTTGAACTT/gatctcctgg

Construct StSUT1/AtSUT2 loop has nucleotides 1 to 749 of StSUT1 (SEQ ID no. 22), nucleotides 843 to 1130 of AtSUT2 (SEQ ID no. 3), and nucleotides 894 to 1548 of StSUT1 (SEQ ID no. 22).

1. Fusion site:

. . . AACGAGCT/tcctttta . . .
. . . AACGAGCT/ccctttta . . .

2. Fusion site:

. . . ctcttacatg/GATCGCGT . . .
. . . ctcttacatg/GATCTCGT . . .

8.2 Functional Analysis of AtSUT2 and Chimeric Proteins

For saccharose uptake tests, yeast strain SEY6210 (Banakaitis (Proc. Natl. Acad. Sci. USA (1986) 83, 9705-9070), which has the corresponding cDNAs in expression vector pDR196,was used. The uptake of 14-C saccharose took place as described in the literature (Weise et al. (2000) Plant Cell 12: 1345-1355). An expression analysis of the proteins in yeast revealed comparable amounts for all of the proteins studied.

It was shown that the saccharose uptake by yeast cells that express AtSUT2 was linear in the first five minutes of the test. During this period, 0.1 nmol saccharose accumulated in 10⁸ cells. The uptake of saccharose by AtSUT2 was significantly higher (p<0.05) than in yeast cells that only expressed the empty vector pDR196. By contrast, the transport rate with StSUT1-expressing cells was 400 times greater than with AtSUT2-expressing cells. Kinetic studies revealed a very low affinity of AtSUT2 for saccharose. Using the Michaelis-Menten equation and a nonlinear regression analysis, a K_{M} value of 11.7±1.2 mM

(Vₘₐₓ=1.5 nmol·min⁻¹ 10⁸ cells⁻¹) was determined for AtSUT2 at a pH of 4. In contrast, StSUT1 had a 10-times lower K_{M} value for saccharose at 1.7 mM (Vₘₐₓ=210.2 nmol·min⁻¹ 10⁸ cells⁻¹).

The saccharose uptake by AtSUT2 was pH-dependent, and the highest uptake rates were measured at a pH of 4.0. The saccharose uptake decreased sharply at alkaline pH values, and at a pH of 6 no further saccharose uptake was measured. To determine the substrate specificity of AtSUT2, the saccharose uptake (1 mM saccharose) was measured competitively with other sugars and sugar alcohols. The tested substrates (saccharose, maltose, isomaltulose, glucomannitol, glucosorbitol, raffinose, galactose, lactose, mannitol, sorbitol, glucose) only the saccharose and to a lesser extent maltose were able to compete significantly with ¹⁴C-saccharose. The saccharose transport by means of AtSUT2 was inhibited by CCCP and by the inhibitor of mitochondrial ATP-formation, antimycin A. These data suggest a proton-coupled transport mechanism for AtSUT2.

8.5 Saccharose Uptake Kinetics in the Saccharose Uptake of Chimeric Proteins

The results for AtSUT2, StSUT1, and the chimeric proteins are shown in the following table:

Table: K_{M} values for saccharose of the saccharose transporters StSUT1 and AtSUT2 as well as of chimeric proteins in which the N-terminal regions or central cytoplasmic loops are exchanged between the two transporters. The values were determined as mean values±standard errors from at least three different measurements. Different letters indicate significant differences (p<0.05).

| | | Vₘₐₓ (nmol | | | |
|---|---|---|---|---|---|
| | K_{M} | saccharose min⁻¹ | | | Membrane |
| Transporter | (nmol L⁻¹) | 10⁸ cells⁻¹) | N-Terminus | Central Loop | Passage |
| AtSUT2 | 11.7 ± 1.2^{a} | 1.5 ± 0.1 | AtSUT2 | AtSUT2 | AtSUT2 |
| AtSUT2/StSUT1- | 6.7 ± 2.0^{bc} | 0.4 ± 0.1 | AtSUT2 | StSUT1 | AtSUT2 |
| loop | | | | | |
| AtSUT2/StSUT1-N | 3.7 ± 1.7^{c} | 0.3 ± 0.05 | StSUT1 | AtSUT2 | AtSUT2 |
| StSTU1/AtSUT2-N | 8.1 ± 1.4^{b} | 72.9 ± 4.9 | AtSUT2 | StSUT1 | StSUT1 |
| StSUT1/AtSUT2- | 1.4 ± 0.3^{c} | 5.5 ± 0.3 | StSUT1 | AtSUT2 | StSUT1 |
| loop | | | | | |
| StSUT1 | 1.7 ± 0.2^{c} | 210.2 ± 5.7 | StSUT1 | StSUT1 | StSUT1 |

The chimeric protein coded by the chimeric construct AtSUT2/StSUT1-N has a significantly lower K_{M} value for saccharose—3.4±1.6 mM (V_{max.}=0.3 nmol·min⁻¹ 10⁸ cells^{31 1})—compared with AtSUT2. By comparison, the chimeric protein coded by chimeric construct StSTU1/AtSUT2-N has a significantly higher K_{M} value for saccharose—8.08±1.4 mM (V_{max.}=72.9 nmol·min⁻¹ 10⁸ cells⁻¹) compared with StSUT1. AtSUT2/StSUT1-loop had a higher K_{M} value (6.75 mM±1.9) (V_{max.}=0.4 nmol·min⁻¹ 10⁸ cells⁻¹) for saccharose, while StSUT1/AtSUT2-loop had a lower Kₘ value (1.4 mM±0.3) (V_{max.}=5.5 nmol·min⁻¹ 10⁸ cells⁻¹) for saccharose.

8.6. Significance of the N-termini of Saccharose Transporters

The following conclusions can be drawn from the expression experiments on chimeric proteins in yeast described above. Replacing the N-terminus of the high-affinity transporter StSUT1 with that of the low-affinity AtSUT2 resulted in an increase of the K_{M} value from 1.7 mM to 8.1 mM (p<0.05). The StSUT1 N-terminus gave a high affinity to AtSUT2 as shown by a K_{M} value that decreased from 11.7 mM to 3.4 mM (p<0.05). Structural differences in the N-terminus between StSUT1 and AtUST2 therefore appear to cause most of the differences in substrate affinity. It is probable that the N-terminus of the saccharose transporter affects the affinity to saccharose as a result of intramolecular interactions with other cytosolic domains or by controlling the position of the first transmembrane passage. This conclusion is not theoretically constrained.

What is claimed is:

1. A process for modifying the saccharide flux or the saccharide concentration in the tissues of a plant, wherein the activity of a saccharide transporter having a high transport capacity for the saccharide and a low affinity to the saccharide is modified by transforming at least one plant cell using at least one vector and by regenerating and obtaining therefrom a plant in whose tissue a modified saccharide flux or a modified saccharide concentration is present, and where the vector has a nucleotide sequence whose expression causes a modification of the transport activity of the saccharide transporter.

2. The process of claim 1, wherein the vector comprises SUT4-coding nucleotide sequences, portions thereof, or a complementary sequence thereof.

3. The process of claim 2, further comprising SUT2-coding nucleotide sequences, portions thereof, or a complementary sequence thereof.

4. The process of claim 2, further comprising SUT1-coding nucleotide sequences, portions thereof, or a complementary sequence thereof.

5. The process of claim 1, wherein the vector comprises SUT2-coding nucleotide sequences, portions thereof, or a complementary sequence thereof.

6. The process of claim 5, further comprising SUT 1-coding nucleotide sequences, portion 1 thereof, or a complementary sequence thereof.

7. The process of claims 1, wherein the vector comprises SUT1-coding nucleotide sequences, portions thereof, or a complementary sequence thereof.

8. The process of claim 7, further comprising SUT2-coding nucleotide sequences and SUT4-coding nucleotide sequences, portion 1 thereof or a complementary sequence thereof.

9. The process of claim 1, wherein the coding nucleotide sequences are cDNA or genomic DNA sequences.

10. The process of claim 1, wherein the plant cell is transformed by at least one vector that produces a leaf-specific overexpression of the coding nucleotide sequence.

11. The process of claim 1, wherein the plant cell is transformed by at least one vector that produces a specific overexpression of the coding nucleotide sequence in guard cells.

12. The process of claim 1, wherein the plant cell is transformed by at least one vector that produces a specific expression or mutagenesis of the coding nucleotide sequences in guard cells and achieves a reduced expression of at least one endogenously present SUT1-, SUT2-, or SUT4-coding nucleotide sequence by means of co-suppression, mutagenesis, RNA-double-strand inhibition, or antisense expression.

13. The process of claim 1, wherein the plant cell is transformed by at least one vector that produces a specific overexpression of the coding nucleotide sequences in sink tissue and/or the parenchyma.

14. The process of claim 1, wherein the plant cell is transformed by at least one vector that produces a specific expression or mutagenesis of the coding nucleotide sequences in sink cells and reduced expression of at least one endogenously present SUT1-, SUT2-, or SUT4-coding nucleotide sequence by means of co-suppression, mutagenesis, RNA-double-strand inhibition, or antisense expression.

15. The process of one of claim 1, wherein the plant cell is transformed by at least one vector that produces a specific expression or mutagenesis of the coding nucleotide sequences in leaves and reduced expression of at least one endogenously present SUT1-, SUT2-, or SUT4-coding nucleotide sequence by means of co-suppression, mutagenesis, RNA-double-strand inhibition, or antisense expression.

16. The process of claim 1, wherein the plant cell is transformed by at least one vector that produces a seed-specific overexpression of the coding nucleotide sequences.

17. The process of claim 1, wherein the plant cell is transformed by at least one vector that produces a specific overexpression of the coding nucleotide sequence in the leaf mesophyll or leaf epidermis.

18. The process of claim 1, wherein the coding nucleotide sequences are under the operative control of at least one regulatory element that produces the expression of an RNA in procaryotic or eucaryotic cells.

19. The process of claim 18, wherein the coding nucleotide sequences in the sense or antisense orientation are under the operative control of at least one regulatory element.

20. The process of claim 19, wherein the regulatory element, of which at least one is present, is a promotor.

21. The process of claim 20, wherein the promotor is GAS, SUC2, SUT1, CaMV35S, ro1C, enhanced PMA4, KAT1, StLS1/L700, PFP, patatin-B33, AAP1, and vicilin promotor.

22. The process of claim 1, wherein the saccharide is saccharose.

23. A nucleic acid molecule, coding a saccharide transporter having a low saccharide affinity and high transport capacity for the saccharide, selected from the group comprising:
a) nucleic acid molecules that comprise the nucleotide sequence shown in SEQ ID NOS: 1, 2, or 27, a portion thereof, or a complementary strand thereof;
b) nucleic acid molecules that encode a protein having the amino acid sequence shown in SEQ ID NOS: 5, 6, or 28, and
c) nucleic acid molecules that hybridize with one of the nucleic acid molecules cited in a) and b).

24. The nucleic acid molecule of claim 23, wherein the saccharide transporter is a saccharose transporter.

25. The nucleic acid molecule of claim 24, wherein the saccharose transporter is SUT4.

26. A nucleic acid molecule coding a regulator or sensor of the saccharide transport, selected from the group comprising:
a) nucleic acid molecules that comprise the nucleotide sequence shown in SEQ ID NOS: 3, 4, 24, 26, or 29, a portion thereof, or a complementary stand thereof;
b) nucleic acid molecules that encode a protein having the amino acid sequence shown in SEQ ID NOS: 7, 8, or 30; and
c) nucleic acid molecules that hybridize with one of the nucleic acid molecules cited in a) and b).

27. The nucleic acid molecule of claim 26, wherein the saccharide transport is the saccharose transport in plants, or a portion thereof.

28. The nucleic acid molecule of claim 27, wherein the saccharose transport is SUT2.

29. A nucleic acid molecule coding at least the N-terminal region of SUT1, shown in SEQ ID NOS: 22 and 25.

30. The nucleic acid molecule of claim 23, wherein the molecule is a DNA or RNA molecule.

31. The nucleic acid molecule of claim 26, wherein the molecule is a DNA or RNA molecule.

32. The nucleic acid molecule of claim 30, wherein the DNA molecule is a cDNA or a genomic DNA.

33. The nucleic acid molecule of claim 31, wherein the DNA molecule is a cDNA or a genomic DNA.

34. A nucleic acid molecule that encodes a chimeric protein, wherein the 5′-terminal area of the coding region of the nucleic acid molecule represents the N-terminal area of the SUT2 protein, and the remainder represents a coding area of a gene that is associated with the metabolism or transport of saccharose.

35. A nucleic acid molecule that encodes a chimeric protein, wherein the 5′-terminal area of the coding region of the nucleic acid molecule represents the N-terminal area of the SUT1 gene, and the remainder represents a coding area of a gene that is associated with the metabolism or transport of saccharose.

36. A nucleic acid molecule that represents a chimeric nucleic acid molecule and that codes for a chimeric protein whose N-terminal area is the N-terminal area of SUT2 and whose remainder is the coding sequence of SUT1.

37. A nucleic acid molecule that represents a chimeric nucleic acid molecule and that encodes a chimeric protein whose N-terminal area is the N-terminal area of SUT1 and whose remainder is the coding sequence of SUT2.

38. A nucleic acid molecule that represents a chimeric nucleic acid molecule and that encodes for a chimeric protein whose central cytoplasmatic domain, which lies between membrane range VI and VII, is coded by SUT2, and whose other areas are coded by a different saccharose transporter gene.

39. The nucleic acid molecule of claim 38, wherein the saccharose transporter gene is SUT1 or SUT4.

40. A vector containing a nucleic acid molecule of claim 23.

41. The vector of claim 40, further containing a saccharide-transporter-coding nucleotide sequence, a portion thereof, or a complementary nucleotide sequence thereof.

42. The vector of claim 40, wherein the nucleic acid molecule is operatively linked to at least one regulatory element that produces the expression of an RNA in procaryotic or eucaryotic cells.

43. The vector of claim 42, wherein the regulatory element is a promotor.

44. The vector of claim 43, wherein the promotor is a member selected from the group consisting of GAS, SUC2,SUT1, CaMV35S, ro1C, enhanced PMA4, KAT1, StLS1/L700, PFP, patatin-B33, AAP1, and vicilin promotor.

45. The vector of claim 40, wherein the nucleic acid molecule, saccharide-transporter-coding nucleotide sequence, or portions thereof, is disposed in an operative antisense orientation relative to the regulatory element, of which at least one is present.

46. A host cell containing the vector of claim 40.

47. The host cell of claim 46, wherein the host is a member selected from the group consisting of a plant cell, a bacteria cell, and a yeast cell.

48. A saccharide transporter having a low saccharide affinity and a high saccharide transport rate, coded by a nucleic acid molecule of claim 23.

49. A protein having the biological activity of a regulator or a sensor of the saccharide transport, coded by a nucleic acid molecule of claim 26.

50. A chimeric protein coded by one of the nucleic acid molecules of claim 34.

51. A transgenic plant cell that is transformed with a nucleic acid molecule of claim 23.

52. A transgenic plant cell that is transformed with a vector of claim 38.

53. The transgenic plant cell of claim 51 that was transformed with a vector containing an SUT/SUC-coding nucleotide sequence, or that descends from such a cell.

54. The transgenic plant cell of claim 53, wherein the SUT/SUC-coding nucleotide sequence is an SUT1-coding nucleotide sequence.

55. A transgenic plant cell whose genome contains at least two stably integrated modified genes from the SUT/SUC gene family.

56. A transgenic plant cell, whose genome contains at least two stably integrated modified genes selected from the group consisting of SUT1/SUT2; SUT1/SUT4, SUT2/SUT4, and SUT1/SUT2/SUT4.

57. A transgenic plant containing at least one plant cell of claim 51.

58. A transgenic plant containing at least one plant cell of claim 53.

59. A transgenic plant containing at least one plant cell of claim 55.

60. A transgenic plant containing at least one plant cell of claim 56.

61. A transgenic plant prepared using the process of claim 1.

62. The transgenic plant of claim 57, wherein the plant is a member selected from the group consisting of graminae, pinidae, magnoliidae, ranunculidae, caryophyllidae, rosidae, asteridae, aridae, liliidae, arecidae, and commelinidae.

63. The transgenic plant of claim 57, wherein the plant is selected from the group consisting of sugar beet, sugar cane, topinambur, arabidopsis, sunflower, tomato, tobacco, corn, barley, wheat, rye, oats, rice, potato, rapeseed, manioc, lettuce, spinach, grapes, apples, coffee, tea, bananas, coconuts, palms, peas, beans, pines, poplar, and eucalyptus.

64. Reproductive or harvest material of a plant of claim 57, containing at least one plant cell transformed with a nucleic acid molecule, coding a saccharide transporter having a low saccharide affinity and high transport capacity for the saccharide, selected from the group comprising:
a) nucleic acid molecules that comprise the nucleotide sequence shown in SEQ ID NOS: 1, 2, or 27, a portion thereof, or a complementary strand thereof;
b) nucleic acid molecules that encode a protein having the amino acid sequence shown in SEQ ID NOS: 5, 6, or 28, and
c) nucleic acid molecules that hybridize with one of the nucleic acid molecules cited in a) and b).

65. The use of a nucleotide sequence of claim 23 for identifying a modulator, in particular an inhibitor, of the saccharide transport in plants, in particular SUT4.

66. The use of a nucleotide sequence of claim 23 to identify an interactor, which is used in turn to affect the saccharide transport.

67. The use of claim 65, wherein the inhibitor inhibits phloem loading in source organs or the unloading in sink organs.

68. The use of a nucleotide sequence of claim 26 to identify a modulator.

69. The use of claim 68, wherein the modulator is an inhibitor of the saccharide transport in plants.

70. The use of claim 69, wherein the saccharide transport is SUT2.

71. The use of claim 68, wherein the inhibitor inhibits the regulation or sensing of the saccharide transport system.

72. The use of a nucleotide sequence of claim 23 as a molecular marker for crossing programs.

73. The use of a 5′-terminal nucleotide sequence of a protein-coding area of a gene from the SUT/SUC gene family to modify the affinity of any given protein with respect to a substrate.

74. The use of claim 73, wherein the protein is from the family of SUT/SUC proteins.

75. The use of claim 73, wherein the substrate is sacharose.

76. The use of claim 72, wherein the gene is a member selected from the group consisting of SUT1, SUT2, and SUT4.

77. The use of claim 73, wherein the N-terminal nucleotide sequence is the nucleotide sequence shown in SEQ ID NO. 24 or 25.

78. The use of the central cytoplasmatic loop of SUT2 for regulation or signal transduction.

79. The use of claim 78, wherein the signal transduction is sugar metabolism.


SEQUENCE LISTING (30 sequences)

SEQ ID NO: 1 - DNA, 1533 bp, Arabidopsis thaliana
atggctacttccgatcaagatcgccgtcacagagccactcgcaaccgtccaccaatacctcgaccctctaattcatcatctcgtcccgttgtacctcctcctcgatcaaaagtttcgaagcgtgtgcttctccgtgtagcttccgtcgcatgcgggattcaattcggatgggcgcttcagctttcgcttctcacaccttacgttcaagagctagggatcccacacgcttgggctagtgtgatttggctttgcggtcctctctctggtttgttcgtgcaaccgctcgttgggcatagtagcgataggtgtactagtaagtacggtcgtcggagaccgtttattgtcgccggagctgtggcgatttctatctctgttatggttattggtcatgcggcggatattggatgggcatttggggatagagaagggaagattaagccgagggcgattgttgcttttgttttagggttttggattcttgatgttgctaataatatgactcaaggtccttgtagagctctcctcgctgatcttactgagaatgataatcgcagaacccgggtggcaaatggctacttctctctctttatggctgttggcaatgttcttggctatgctactggatcatacaatggttggtacaagatcttcacttttacgaagacagttgcatgtaatgtggaatgtgccaatctcaagtctgccttctacatagatgttgtctttattgcaataactacgatcctaagcgtttcagcggctcatgaggtgcctcttgcttcattgacttctgaagcacatgggcaaaccagtggaacagacgaagcttttctttctgagatatttggaactttcagatattttccaggaaatgtttggataatcttgcttgttacagcattgacatggattggttggtttccatttattctgtttgatactgattggatgggtcgagagatctatggcggtgaaccgaacatagggacttcatatagtgctggggtcagtatgggtgcacttggtttgatgttgaattctgtttttcttggaatcacttcggtgctcatggagaaactttgcagaaagtggggggctggttttgtttggggaatatcaaatatcttaatggctatttgctttcttggaatgataatcacctcatttgttgcgtctcaccttggctacattggccatgaacaacctcctgccagcatcgtgtttgctgctgtgttaatctttacaattctgggcattccattggcgataacttacagcgtcccatatgcgttgatttccatacgtattgaatccctgggcctaggtcaaggcttatctttgggtgtgctaaatttggcgatagtcatcccacaggtaattgtgtctgttggcagtggcccatgggatcaactgtttggaggtgggaattcaccggcacttgcagtaggagcagctacaggcttcattggcggaattgtagctatcttggctcttccacggacaaggattcagaagcccatccctctcccatga

SEQ ID NO: 2 - DNA, 1503 bp, Lycopersicon esculentum
atgccggagatagaaaggcatagaacaaggcataaccgaccggcgattcgagaaccggtgaaaccgagagtaccactgagactattgttccgagtagcttcggttgccggtggaattcaattcggttgggcgttacaactatcactgctcacaccttatgtgcaagagcttggaataccgcatgcttgggcgagcataatatggctctgtggaccgctttcaggtttactggttcagcctttagtaggtcacatgagtgacaagtgcacaagtcggttcggtcgtcggcgcccgtttattgtcgccggagcagtatcgatcatgattgcggtgttgattatcggtttctccgctgatattggatggcttttaggtgatcgaggtgaaataaaagtgcgtgctatagcggcgtttgtcgtagggttttggcttctcgatgttgccaataatatgactcaaggaccttgcagagctctgcttgctgatcttacacaaaaggatcatagaagaacccgggtagcaaatgcatatttttccttatttatggccattggtaacatccttggctttgctactggatcttacagtggctggttcaagatcttcccttttactctcaatactgcatgcaccatcaactgtgccaatctaaaggctgcttttattatcgacattatttttattgcaacaactacatgcattagcatatcagcggccaatgagcagcctctagatcccagtcgtggttcctctcataccagagaagagattggcgaatcaagccatggtcaagaagaagcttttctctgggagttgtttggaattttcaagtatttcccaggtgttgtttgggtgatcctgcttgtcactgccctgacatggattggatggtttccgtttcttttgttcgatactgactggtttggtcgagaaatttatggcggtgaaccaaatgatggaaagaattatagtgcaggagtgcgaatgggttcattgggtctaatgttgaattctgtgcttcttggactaacttcattgttcatggagaagctctgtcgaaaatggggtgctggtttcacatggggagtttcaaacgtggtcatgtctctctgttttatagccatgcttataattactgctgttaggagtaacatagacattggccagggtcttccaccggatggcattgtgattgctgcgctggttgtattttctattcttgggatcccactagctataacatacagtgttccatatgctttagtatcctcaaggattgatgctcttgggcttggacaaggcttgtcaatgggtgtgctgaacctggcaattgtgttcccacagattgtggtttctctgggaagtgggccatgggatgagttatttggtggaggcaattcaccagcctttgttgtggctgcgctttcagcatttgctggtggacttatagccatcttggcgattcctcgaacacgggttgagaaacccaagatctttgcatga

SEQ ID NO: 3 - DNA, 1785 bp, Arabidopsis thaliana
atgagtgactcggtgtcgatctcggttccgtataggaatttgaggaaggaaattgaacttgagacggtcaccaagcatcgtcaaaacgaatctggttcttcgtcgttctctgaatctgcttctccttcgaatcattctgattcggctgatggtgaatctgtgtcgaagaattgtagtttagtgacgttggttcttagttgtacagttgccgctggagttcaatttggttgggcattgcaactttctcttcttactccttatattcagacccttggaatatcgcatgctttttcttcgtttatttggctgtgcggcccaattacaggccttgtggtccagccttttgttggcatttggagtgataaatgtacttcaaagtatggaagaagacgaccatttattctagttggatcattcatgatctcaatagcagtgataataatcggattttctgcagacattgggtatctgttaggagattcaaaggaacattgcagtactttcaaaggcacacgaaccagggcagctgttgtctttatcattgggttttggttgttggatctagcaaacaatacagtacagggacctgctcgtgctcttctagctgatctatcaggtcctgatcagcggaatactgcaaatgctgtgttctgcttgtggatggctattgggaacatccttgggttttctgccggtgctagcggaaaatggcaagaatggttcccttttttaactagtagagcatgttgtgctgcatgtggaaatctcaaagcagcgtttcttcttgcagtggtctttctcactatatgtactcttgtcacaatctattttgctaaagagattccttttacaagcaacaagcccacccgcatacaagattctgcacctttgttggatgatctccagtccaaaggccttgagcattcaaaattaaataatggtactgccaatggaatcaagtatgagagagtggaacgtgatacggatgaacagtttggcaattcagagaatgagcatcaagatgagacctacgttgatggccctggatctgttttagtgaatttgctaactagtttaaggcatttgccaccggctatgcactcagttcttatcgtcatggctcttacatggttatcctggttccccttctttctgttcgatacagattggatgggaagagaagtttaccatggggatccaacaggagatagtttgcatatggaactctatgatcaaggtgtacgtgaaggtgcacttggtttgctactaaactctgttgttcttgggatcagctcatttctcattgaaccaatgtgtcagcggatgggtgctcgggttgtatgggctttgagcaattttactgtatttgcctgcatggcgggaacagctgtaatcagcttgatgtctctcagtgatgacaaaaatggaattgaatacataatgcgtggaaacgaaacaacaagaaccgcagccgtaatcgtttttgcactccttggttttcccctagctatcacatacagtgtccctttctctgtcacagcagaagtcactgctgattccggtggcggtcaaggtttggctataggagtgttgaatctcgcaatcgttattccccagatgatagtatcacttggagcgggtccatgggatcaattgtttggaggaggaaacttaccggcgtttgttttggcgtctgttgctgctttcgctgctggagttattgcattgcaaaggcttcccacgctatcgagttctttcaagtccaccggtttccacatcggctaa

SEQ ID NO: 4 - DNA, 1355 bp, Lycopersicon esculentum
ctgcagacataggatacttattgggggacacaaaagagcattgcagcactttcaaaggcactcgctcaagagcagccattgtatttgtcgttgggttttggatgctcgatcttgctaataatactgtgcagggtccggctcgagctcttttggcagatttgtcaggtcctgatcaaagaaataccgcaaatgctgtgttctgctcctggatggctgttggaaacattcttggattttctgctggagccagtggaggttggcacagatggtttccgtttttgacaaatagagcttgttgtgagccatgtggaaatctcaaagcagcattcttagttgcagtggtctttctaactctctgcacgttagtaactctctacttcgccaatgaagtcccactgtcacccaagcaatataaacgcttgtcagattctgctcctctcttggatagtcctcagaatactggctttgacctttctcaatcaaaaagggagttgcagtctgtaaatagtgtagcaaataatgaatctgagatgggtcgtgtagcagataatagtccaaagaatgaagaacagagacctgacaaggatcaaggtgatagctttgctgatagccctggagcagttttggtcaatctgttgaccagcttacgtcatttgcctcccgcaatgcattcggttctcattgtcatggctctgacttggttgccctggtttccctttttcctttttgacacggattggatggggagagaagtctatcatggggacccgaaaggagaagcagatgaagtaaatgcatataaccaaggtgtcagagaaggtgcatttggtttgctattgaattctgttgttcttggcgttagctcctttcttattgagccaatgtgcaagtggattggttctagacttgtttgggctgtgagcaacttcattgtatttgtctgcatggcctgcaccgctatcattagcgtggtttccatcagtgcacatacggagggagtccaacatgtgattggtgctactaaatcaactcaaattgctgctttggttgttttctctcttcttggcattcctcttgctgtaacttacagtgtccctttctctatcacagcagagttgacagctgacgctggtggtggtcaagggttggcaataggagtcctgaatcttgcaatcgttttacctcagatggttgtctcgcttggtgccggtccatgggatgctttatttggtggaggaaacataccggcatttgtcttagcatctttagctgcacttgctgctggaatttttgctatgctcagactaccaaatttatcaagtaatttcaaatcaactggcttccattttggttga

SEQ ID NO: 5 - protein, 510 aa, Arabidopsis thaliana
MATSDQDRRHRATRNRPPIPRPSNSSSRPVVPPPRSKVSKRVLLRVASVACGIQFGWALQLSLLTPYVQELGIPHAWASVIWLCGPLSGLFVQPLVGHSSDRCTSKYGRRRPFIVAGAVAISISVMVIGHAADIGWAFGDREGKIKPRAIVAFVLGFWILDVANNMTQGPCRALLADLTENDNRRTRVANGYFSLFMAVGNVLGYATGSYNGWYKIFTFTKTVACNVECANLKSAFYIDVVFIAITTILSVSAAHEVPLASLTSEAHGQTSGTDEAFLSEIFGTFRYFPGNVWIILLVTALTWIGWFPFILFDTDWMGREIYGGEPNIGTSYSAGVSMGALGLMLNSVFLGITSVLMEKLCRKWGAGFVWGISNILMAICFLGMIITSFVASHLGYIGHEQPPASIVFAAVLIFTILGIPLAITYSVPYALISIRIESLGLGQGLSLGVLNLAIVIPQVIVSVGSGPWDQLFGGGNSPALAVGAATGFIGGIVAILALPRTRIQKPIPLP

SEQ ID NO: 6 - protein, 500 aa, Lycopersicon esculentum
MPEIERHRTRHNRPAIREPVKPRVPLRLLFRVASVAGGIQFGWALQLSLLTPYVQELGIPHAWASIIWLCGPLSGLLVQPLVGHMSDKCTSRFGRRRPFIVAGAVSIMIAVLIIGFSADIGWLLGDRGEIKVRAIAAFVVGFWLLDVANNMTQGPCRALLADLTQKDHRRTRVANAYFSLFMAIGNILGFATGSYSGWFKIFPFTLNTACTINCANLKAAFIIDIIFIATTTCISISAANEQPLDPSRGSSHTREEIGESSHGQEEAFLWELFGIFKYFPGVVWVILLVTALTWIGWFPFLLFDTDWFGREIYGGEPNDGKNYSAGVRMGSLGLMLNSVLLGLTSLFMEKLCRKWGAGFTWGVSNVVMSLCFIAMLIITAVRSNIDIGQGLPPDGIVIAALVVFSILGIPLAITYSVPYALVSSRIDALGLGQGLSMGVLNLAIVFPQIVVSLGSGPWDELFGGGNSPAFVVAALSAFAGGLIAILAIPRTRVEKPKIFA

SEQ ID NO: 7 - protein, 594 aa, Arabidopsis thaliana
MSDSVSISVPYRNLRKEIELETVTKHRQNESGSSSFSESASPSNHSDSADGESVSKNCSLVTLVLSCTVAAGVQFGWALQLSLLTPYIQTLGISHAFSSFIWLCGPITGLVVQPFVGIWSDKCTSKYGRRRPFILVGSFMISIAVIIIGFSADIGYLLGDSKEHCSTFKGTRTRAAVVFIIGFWLLDLANNTVQGPARALLADLSGPDQRNTANAVFCLWMAIGNILGFSAGASGKWQEWFPFLTSRACCAACGNLKAAFLLAVVFLTICTLVTIYFAKEIPFTSNKPTRIQDSAPLLDDLQSKGLEHSKLNNGTANGIKYERVERDTDEQFGNSENEHQDETYVDGPGSVLVNLLTSLRHLPPAMHSVLIVMALTWLSWFPFFLFDTDWMGREVYHGDPTGDSLHMELYDQGVREGALGLLLNSVVLGISSFLIEPMCQRMGARVVWALSNFTVFACMAGTAVISLMSLSDDKNGIEYIMRGNETTRTAAVIVFALLGFPLAITYSVPFSVTAEVTADSGGGQGLAIGVLNLAIVIPQMIVSLGAGPWDQLFGGGNLPAFVLASVAAFAAGVIALQRLPTLSSSFKSTGFHIG

SEQ ID NO: 8 - protein, 450 aa, Lycopersicon esculentum
ADIGYLLGDTKEHCSTFKGTRSRAAIVFVVGFWMLDLANNTVQGPARALLADLSGPDQRNTANAVFCSWMAVGNILGFSAGASGGWHRWFPFLTNRACCEPCGNLKAAFLVAVVFLTLCTLVTLYFANEVPLSPKQYKRLSDSAPLLDSPQNTGFDLSQSKRELQSVNSVANNESEMGRVADNSPKNEEQRPDKDQGDSFADSPGAVLVNLLTSLRHLPPAMHSVLIVMALTWLPWFPFFLFDTDWMGREVYHGDPKGEADEVNAYNQGVREGAFGLLLNSVVLGVSSFLIEPMCKWIGSRLVWAVSNFIVFVCMACTAIISVVSISAHTEGVQHVIGATKSTQIAALVVFSLLGIPLAVTYSVPFSITAELTADAGGGQGLAIGVLNLAIVLPQMVVSLGAGPWDALFGGGNIPAFVLASLAALAAGIFAMLRLPNLSSNFKSTGFHFG

SEQ ID NO: 9 - DNA, 29 bp, Arabidopsis thaliana
gatgcactcgaaatcagccaattttagac

SEQ ID NO: 10 - DNA, 28 bp, Agrobacterium tumefaciens
tcatgggagagggatgggcttctgaatc

SEQ ID NO: 11 - DNA, 28 bp, Arabidopsis thaliana
atggctacttccgatcaagatcgccgtc

SEQ ID NO: 12 - DNA, 29 bp, Lycopersicon esculentum
gactctgcagcgagaaatggctacttccg

SEQ ID NO: 13 - DNA, 29 bp, Lycopersicon esculentum
taacctgcaggagaatctcatgggagagg

SEQ ID NO: 14 - DNA, 29 bp, Lycopersicon esculentum
gagactgcagatgccggagatagaaaggc

SEQ ID NO: 15 - DNA, 33 bp, Lycopersicon esculentum
tatgacagcggccgctcatgcaaagatcttggg

SEQ ID NO: 16 - protein, 23 aa, Lycopersicon esculentum
MPEIERHRTRHNRPAIREPVKPR

SEQ ID NO: 17 - protein, 22 aa, Lycopersicon esculentum
GSSHTGEEIDESSHGQEEAFLW

SEQ ID NO: 18 - DNA, 19 bp, Arabidopsis thaliana
atggctgaccagatttgac

SEQ ID NO: 19 - DNA, 18 bp, Arabidopsis thaliana
gtttcatattaatttcac

SEQ ID NO: 20 - DNA, 30 bp, Arabidopsis thaliana
tacgagaattcgatctgtgtgttgaggacg

SEQ ID NO: 21 - DNA, 27 bp, Arabidopsis thaliana
agaggctcgagtggtcaaaaagaatcg

SEQ ID NO: 22 - DNA, 1548 bp, Solanum tuberosum
atggagaatggtacaaaaagagaaggtttagggaaacttacagtttcatcttctctacaagttgaacagcctttagcaccatcaaagctatggaaaattatagttgtagcttccatagctgctggtgttcaatttggttgggctcttcagctctctttgcttacaccttatgttcaattgctcggaattcctcataaatttgcctcttttatttggctttgtggaccgatttctggtatgattgttcagccagttgtcggctactacagtgataattgctcctcccgtttcggtcgccgccggccattcattgccgccggagctgcacttgttatgattgcggttttcctcatcggattcgccgccgaccttggtcacgcctccggtgacactctcggaaaaggatttaagccacgtgccattgccgttttcgtcgtcggcttttggatccttgatgttgctaacaacatgttacagggcccatgcagagcactactggctgatctctccggcggaaaatccggcaggatgagaacagcaaatgcttttttctcattcttcatggccgtcggaaacattctggggtacgccgccggttcatattctcacctctttaaagtattccccttctcaaaaaccaaagcctgcgacatgtactgcgcaaatctgaagagttgtttcttcatcgctatattccttttactcagcttaacaaccatagccttaaccttagtccgggaaaacgagctcccggagaaagacgagcaagaaatcgacgagaaattagccggcgccggaaaatcgaaagtaccgtttttcggtgaaatttttggggctttgaaagaattacctcgaccgatgtggattcttctattagtaacctgtttgaactggatcgcgtggtttccctttttcttatacgatacagattggatggctaaggaggttttcggtggacaagtcggtgatgcgaggttgtacgatttgggtgtacgcgctggtgcaatgggattactgttgcaatctgtggttctagggtttatgtcacttggggttgaattcttagggaagaagattggtggtgctaagaggttatggggaattttgaactttgttttggctatttgcttggctatgaccattttggtcaccaaaatggccgagaaatctcgccagcacgaccccgccggcacacttatggggccgacgcctggtgttaaaatcggtgccttgcttctctttgccgcccttggtattcctcttgcggcaacttttagtattccatttgctttggcatctatattttctagtaatcgtggttcaggacaaggtttgtcactaggagtgctcaatcttgcaattgttgtaccacagatgttggtgtcactagtaggagggccatgggatgatttgtttggaggaggaaacttgcctggatttgtagttggagcagttgcagctgccgcgagcgctgttttagcactcacaatgttgccatctccacctgctgatgctaagccagcagtcgccatgggcggtttccattaa

SEQ ID NO: 23 - protein, 515 aa, Solanum tuberosum
MENGTKREGLGKLTVSSSLQVEQPLAPSKLWKIIVVASIAAGVQFGWALQLSLLTPYVQLLGIPHKFASFIWLCGPISGMIVQPVVGYYSDNCSSRFGRRRPFIAAGAALVMIAVFLIGFAADLGHASGDTLGKGFKPRAIAVFVVGFWILDVANNMLQGPCRALLADLSGGKSGRMRTANAFFSFFMAVGNILGYAAGSYSHLFKVFPFSKTKACDMYCANLKSCFFIAIFLLLSLTTIALTLVRENELPEKDEQEIDEKLAGAGKSKVPFFGEIFGALKELPRPMWILLLVTCLNWIAWFPFFLYDTDWMAKEVFGGQVGDARLYDLGVRAGAMGLLLQSVVLGFMSLGVEFLGKKIGGAKRLWGILNFVLAICLAMTILVTKMAEKSRQHDPAGTLMGPTPGVKIGALLLFAALGIPLAATFSIPFALASIFSSNRGSGQGLSLGVLNLAIVVPQMLVSLVGGPWDDLFGGGNLPGFVVGAVAAAASAVLALTMLPSPPADAKPAVAMGGFH

SEQ ID NO: 24 - DNA, 239 bp, Arabidopsis thaliana
atgagtgactcggtgtcgatctcggttccgtataggaatttgaggaaggaaattgaacttgagacggtcaccaagcatcgtcaaaacgaatctggttcttcgtcgttctctgaatctgcttctccttcgaatcattctgattcggctgatggtgaatctgtgtcgaagaattgtagtttagtgacgttggttcttagttgtacagttgccgctggagttcaatttggttgggcattgca

SEQ ID NO: 25 - DNA, 149 bp, Solanum tuberosum
atggagaatggtacaaaaagagaaggtttagggaaacttacagtttcatcttctctacaagttgaacagcctttagcaccatcaaagctatggaaaattatagttgtagcttccatagctgctggtgttcaatttggttgggctcttca

SEQ ID NO: 26 - DNA, 288 bp, Arabidopsis thaliana
tccttttacaagcaacaagcccacccgcatacaagattctgcacctttgttggatgatctccagtccaaaggccttgagcattcaaaattaaataatggtactgccaatggaatcaagtatgagagagtggaacgtgatacggatgaacagtttggcaattcagagaatgagcatcaagatgagacctacgttgatggccctggatctgttttagtgaatttgctaactagtttaaggcatttgccaccggctatgcactcagttcttatcgtcatggctcttacatg

SEQ ID NO: 27 - DNA, 1503 bp, Solanum tuberosum; identical to SEQ ID NO: 2

SEQ ID NO: 28 - protein, 500 aa, Solanum tuberosum; identical to SEQ ID NO: 6

SEQ ID NO: 29 - DNA, 1818 bp, Solanum tuberosum
atggatgcggtatcgatcagagtaccgtataagaatctgaagcagcaggaagtggaattaactaatgttgatgaatcacggtttacacagttggagatccgtagtgattcctcatctcctagggcttctaatggagaaatgaatgattctcatctacctcttcctcctccgcctgtacgcaacagtttgcttaccttgattcttagttgcaccgtcgctgccggtgttcagtttggatgggctttgcaactatctctccttacaccttatattcagacacttggcatagagcatgccttctcttcttttatctggctatgcggtcctattactggccttgtggtacaaccttgtgtaggtatatggagtgataaatgtcattctaaatatggcagaagaaggcctttcatttttattggagctgtcatgatctctattgctgtgataattatcgggttttctgctgcagacataggatacttattgggggacacaaaagagcattgcagcactttcaaaggcactcgctcaagagcagccattgtatttgtcgttgggttttggatgctcgatcttgctaataatactgtgcagggtccggctcgagctcttttggcagatttgtcaggtcctgatcaaagaaataccgcaaatgctgtgttctgctcctggatggctgttggaaacattcttggattttctgctggagccagtggaggttggcacagatggtttccgtttttgacaaatagagcttgttgtgagccatgtggaaatctcaaagcagcattcttagttgcagtggtctttctaactctctgcacgttagtaactctctacttcgccaatgaagtcccactgtcacccaagcaatataaacgcttgtcagattctgctcctctcttggatagtcctcagaatactggctttgacctttctcaatcaaaaagggagttgcagtctgtaaatagtgtagcaaataatgaatctgagatgggtcgtgtagcagataatagtccaaagaatgaagaacagagacctgacaaggatcaaggtgatagctttgctgatagccctggagcagttttggtcaatctgttgaccagcttacgtcatttgcctcccgcaatgcattcggttctcattgtcatggctctgacttggttgccctggtttccctttttcctttttgacacggattggatggggagagaagtctatcatggggacccgaaaggagaagcagatgaagtaaatgcatataaccaaggtgtcagagaaggtgcatttggtttgctattgaattctgttgttcttggcgttagctcctttcttattgagccaatgtgcaagtggattggttctagacttgtttgggctgtgagcaacttcattgtatttgtctgcatggcctgcaccgctatcattagcgtggtttccatcagtgcacatacggagggagtccaacatgtgattggtgctactaaatcaactcaaattgctgctttggttgttttctctcttcttggcattcctcttgctgtaacttacagtgtccctttctctatcacagcagagttgacagctgacgctggtggtggtcaagggttggcaataggagtcctgaatcttgcaatcgttttacctcagatggttgtctcgcttggtgccggtccatgggatgctttatttggtggaggaaacataccggcatttgtcttagcatctttagctgcacttgctgctggaatttttgctatgctcagactaccaaatttatcaagtaatttcaaatcaactggcttccattttggttga

SEQ ID NO: 30 - protein, 605 aa, Solanum tuberosum
MDAVSIRVPYKNLKQQEVELTNVDESRFTQLEIRSDSSSPRASNGEMNDSHLPLPPPPVRNSLLTLILSCTVAAGVQFGWALQLSLLTPYIQTLGIEHAFSSFIWLCGPITGLVVQPCVGIWSDKCHSKYGRRRPFIFIGAVMISIAVIIIGFSAADIGYLLGDTKEHCSTFKGTRSRAAIVFVVGFWMLDLANNTVQGPARALLADLSGPDQRNTANAVFCSWMAVGNILGFSAGASGGWHRWFPFLTNRACCEPCGNLKAAFLVAVVFLTLCTLVTLYFANEVPLSPKQYKRLSDSAPLLDSPQNTGFDLSQSKRELQSVNSVANNESEMGRVADNSPKNEEQRPDKDQGDSFADSPGAVLVNLLTSLRHLPPAMHSVLIVMALTWLPWFPFFLFDTDWMGREVYHGDPKGEADEVNAYNQGVREGAFGLLLNSVVLGVSSFLIEPMCKWIGSRLVWAVSNFIVFVCMACTAIISVVSISAHTEGVQHVIGATKSTQIAALVVFSLLGIPLAVTYSVPFSITAELTADAGGGQGLAIGVLNLAIVLPQMVVSLGAGPWDALFGGGNIPAFVLASLAALAAGIFAMLRLPNLSSNFKSTGFHFG